(19) United States
(12) Patent Application Publication
(10) Pub. No.: US 2004/0045046 A1
(43) Pub. Date: Mar. 4, 2004

(54) Transgenic mammals expressing polyglutamine

(76) Inventors: Huda Y. Zoghbi, Houston, TX (US); Kei Watase, Houston, TX (US); Christopher J. Cummings, Brookline, MA (US)

Correspondence Address: PALMER & DODGE, LLP, KATHLEEN M. WILLIAMS, 111 HUNTINGTON AVENUE, BOSTON, MA, 02199, US

(21) Appl. No.: 10/459,188

(22) Filed: Jun. 11, 2003

Related U.S. Application Data
(60) Provisional application No. 60/387,939, filed on Jun. 11, 2002.

(51) Int. Cl. A01K 67/027

(52) U.S. Cl. 800/18

(57) ABSTRACT

The present invention utilizes a “knock-in” approach to provide a transgenic mammal containing integrated into its genome a repeating nucleotide sequence encoding a polyglutamine comprising at least about 154, preferably at least about 160, 200, 300, 400, 500, and up to 600 or more contiguous glutamine residues. The transgenic mammals normally display observable phenotypic changes. As such, they may serve as a model for disease processes in humans for such diseases as spinobulbar muscular atrophy (SBMA), Huntington's disease (HD), dentatorubral pallidoluysian atrophy (DRPLA), and the spinocerebellar ataxias types 1, 2, 3, 6, 7, and 17. As a result of displaying pathology indicative of a human disease, the efficacy of an agent for treating human disease may be tested in the transgenic mammals.

### PRIORITY

This application claims priority to U.S. Provisional Application Serial No. 60/387,939, filed Jun. 11, 2002, the contents of which are incorporated herein in their entirety.

### FIELD OF THE INVENTION

The present invention relates to novel transgenic non-human mammals. In particular, it relates to transgenic non-human mammals that express a protein involved in a neurological disorder or a protein having a polyglutamine region that mediates a disorder entirely or in part. The present invention also features transgenes and genetic constructs useful in the preparation of transgenic mammals, and methods of screening for biologically active agents, including those useful in the treatment of a neurological disorder.

### BACKGROUND

Spinocerebellar ataxia type I (SCA1) is a dominantly inherited late-onset neurodegenerative disorder characterized by progressive ataxia, dysarthria and swallowing difficulties (Zoghbi et al., (1995) Semin. Cell Biol. 6, 29-35), and is one of nine established neurodegenerative diseases who's etiology is linked to the expansion of a CAG repeat the encodes for polyglutamine in the respective disease proteins. The other diseases include SCA2, 3, 6, 7, and 17, as well as Huntington's disease (HD), spinobulbar muscular atrophy (SBMA) and dentatorubralpallidoluysian atrophy (DRPLA) (Zoghbi and Orr, 2000, Ann. Rev. Neurosci. 23:217). The onset of SCA1 clinical symptoms occurs usually in the third or fourth decade of life, although juvenile cases have been observed (Zoghbi, H. Y., et al. (1998) Ann. Neurol., 23, 580-584). The disease worsens progressively over 10 to 15 years, leading eventually to death due to bulbar dysfunction. Neuropathologically, SCA1 is characterized by selective degeneration of cerebellar Purkinje cells and neurons of the inferior olive and brainstem.

The mutation underlying SCA1 is an expansion of a CAG trinucleotide repeat, encoding a polyglutamine tract in the open reading frame of the SCA1 transcript (Orr et al., (1993) Nature Genet. 4, 221-226). In the normal population, the repeat is polymorphic, with allele sizes ranging from 6 to 44 CAG units, and it is interrupted by at least one CAT triplet. Conversely, SCA1 chromosomes carry uninterrupted CAG tracts ranging in size from 40 to 83 repeat units (Chung, M. Y., et al. (1993) Nature Genet. 5, 254-258; Ranum et al., (1994) Am. J. Hum. Genet. 55, 244-252). Although alleles in the normal size range are relatively stable on germline transmission, expanded alleles change size in the majority of the parent-to-offspring transmissions (Chung, et al. (1993) Nature Genet., 5, 254-258; Ranum et al., (1994) Am. J. Hum. Genet, 55, 244-252). This results in an earlier manifestation of the clinical symptoms in subsequent generations of SCA1 families, a phenomenon known as anticipation. There is an inverse correlation between the size of the expanded alleles and age of disease onset (Orr et al., (1993) Nature Genet. 4, 221-226).

The SCA1 gene encodes a 100 kDa protein of unknown function termed ataxin-1 and is widely expressed in the central nervous system and peripheral tissues (Banfi et al., (1994) Nature Genet., 7, 513-520). Immunohistochemical studies showed that ataxin-1 localizes predominantly to the nucleus of neuronal cells and in the cytoplasm of other cell types (Servadio et al. (1995) Nature Genet., 10, 94-98). In SCA1 patients, ataxin-1 accumulates in single ubiquitin-positive nuclear inclusions which are present in affected neurons (Skinner et al., (1997) Nature, 389, 971-974).

One of the most fruitful approaches to studying polyglutamine diseases has been to generate transgenic mouse models expressing truncated or full-length cDNAs encoding the mutant protein in neurons (Lin et al., (1999) Neuron 24 499-502; Gusella et al. (2000) Nature Rev. Neurosci, 1, 109-115). Most of these transgenic mice overexpress the mutant protein either under neuron-specific or ubiquitous promoters, which is enough to reproduce various aspects of the human neurological phenotypes, including aggregate formation in neurons. The role of these aggregates in the disease process is controversial although data suggest that the aggregates themselves are not necessary for initiating disease (Klement et al., (1998) Cell, 95, 441-53). Though their role in pathogenesis is unclear, much effort has been directed toward interventions that seek to eradicate these deposits.

Numerous lines of transgenic or knock-in mice carrying CAG repeat expansions have been created to reproduce intergenerational repeat instability. Among these are SCA1 cDNA CAG82 transgenic mice (Kaytor et al., (1997) Hum. Mol. Genet. 6, 2135-2159), Hdh41Q80 knock-in mice (Shelbourne et al. (1999) Hum. Mol. Genet. 8, 763-774), HdhQI8-Qlll knock-in mice (Wheeler et al., (1999) Hum. Mol. Genet. 8, 115-122), Huntington exon I transgenic mice (Mangiarini et al., (1997) Nature Genet. 15, 197-200; Lin et al., (2001) Human Mol. Genet. 10(2), 137-144), androgen receptor yeast artificial chromosome (YAQ transgenic mice (CAG45) (La Spada et al. (1998) Hum. Mol. Genet. 7, 959-967) and DRPLA YAC transgenic mice (CAG76 78) (Sato et al. (1999) Hum. Mol. Genet. 8, 99-106).

Burright et al. generated transgenic animals that overexpress mutant ataxin-1 transcripts under the control of the Purkinje cell pcp2 promoter (Burright et al., (1995) Cell 82, 937-948; (Lorenzetti et al., (2000) Hum. Mol. Genet. 9, 779-785). One of these transgenic lines, the B05 line, expresses a full-length mutant ataxin-1 mRNA with 82 CAG repeats (82Q) at around 50-100 times endogenous levels in Purkinje cells after P10 and develop the ataxia typical of human SCA1. These mice allowed the discovery that several specific genes are downregulated in Purkinje cells before any detectable pathological changes (Lin et al., (2000) Nat Neurosci, 3, 157-163). This suggests that mutant ataxin-1 may initiate the disease process by altering gene expression. Studies of the B05 mice also provided early evidence that misfolding and impaired degradation of mutant ataxin-1 might underlie SCA1 pathogenesis (Cummings et al., (1998) Nat. Genet. 19, 148-154). Recent studies indicate that the disease phenotype of B05 mice is aggravated by dysfunction of the ubiquitin-proteosome (UPP) pathway (Cummings et al., (1999) Neuron, 24, 879-892) but suppressed by overexpression of inducible Hsp70 chaperone (Cummings et al., (2001) Hum. Mol. Genet. 10, 1511-1518).

The B05 mice has been useful for disclosing disease mechanisms, and show the phenotype associated with dysfunctional Purkinje cells. These mice live a normal lifespan without demonstrating significant phenotype changes. This makes direct comparison with human SCA1 difficult since human SCA1 patients develop cognitive impairment as the disease progresses, and the final stage of the disease is quite complex, causing patients to die early.

### SUMMARY OF THE INVENTION

The present invention utilizes a “knock-in” approach to provide a transgenic mammal (referred to herein as a “knock-in mammal”) containing integrated into its genome a repeating nucleotide sequence encoding a polyglutamine comprising at least about 154 contiguous codons encoding glutamine. The number of contiguous glutamines may be at least about 154, 160, 200, 300, 400, 500, and up to 600 or more contiguous glutamine residues. A genome containing a repeating sequence encoding a polyglutamine comprising at least 154 codons encoding glutamine in succession has been shown especially useful for providing a model for spinocerebellar ataxias type 1 and 7. One such sequence encoding glutamine is preferably CAG, though due to the degeneracy of the genetic code, other nucleotide sequences may also encode glutamine. CAA is one such additional codon sequence.

The knock-in mammals according to the present invention normally exhibit phenotypic changes that may be observed. Such phenotypic changes include clinical symptoms such as motor incoordination, ataxia, cognitive deficits, muscle wasting, premature death, progressive Purkinje cell degeneration, and age related hippocampal synaptic dysfunction, and are described more fully below. Additionally, the transgenic mammals according to the invention may demonstrate accumulation of a protein comprising at least 154 and preferably about 160, 180, 200, 250, 270, 290, 300, 310, 330, 350, 400, 500, and up to 600 or more glutamines in succession in neurons. Any mammal excluding humans is within the scope of the present invention. However, by way of example, the knock-in mammals may be mice, guinea pigs, rabbits, pigs, sheep, cows, goats or horses.

The knock-in mammals of the present invention may serve as a model for disease processes in humans. Hence, the transgenic mammals may demonstrate clinical symptoms of a disease such as spinobulbar muscular atrophy (SBMA), Huntington's disease (HD), dentatorubral pallidoluysian atrophy (DRPLA), and the spinocerebellar ataxias types 1, 2, 3, 6, 7, and 17.

The transgenic mammals feature codons encoding polyglutamine in succession that are introduced into the coding region for a protein involved in pathogenesis of a corresponding mammalian disease as described below. Such codons encoding glutamine in succession may be introduced in a gene encoding an ataxin, for instance an ataxin-1 gene such as Sca1. Such codons may be introduced into the genome of the mammal by any operable construct, for instance by all or part of an Sca1 exon 8 or by a construct comprising a BamHI Xho1 fragment. Alternatively, the gene encoding the protein involved in the pathogenesis of a corresponding mammalian disease may be completely replaced with a construct containing most or all of the coding region having incorporated therein codons encoding a polyglutamine. In order to produce the gene constructs used in the invention, recombinant DNA and cloning methods which are well known to those skilled in the art may be utilized (See, e.g. Sambrook et al., (1989) Molecular Cloning, A Laboratory Manual, 3rd ed. (Cold Spring Harbor Laboratory)).

In a second aspect, the present invention features a method for testing the efficacy of an agent for treating a disease characterized by the abnormal presence of a polyglutamine sequence in a peptide or protein. The method generally features administering an agent to a knock-in mammal of the invention and monitoring the mammal to determine the effect of the agent on one or more phenotypic characteristics. The knock-in mammals of the present invention normally demonstrate one or more clinical symptoms such as motor incoordination, ataxia, cognitive deficits, muscle wasting, premature death, progressive Purkinje cell degeneration, and age related hippocampal synaptic dysfunction. Such symptoms may be indicative or even diagnostic of a disease such as spinobulbar muscular atrophy (SBMA), Huntington's disease (HD), dentatorubral pallidoluysian atrophy (DRPLA), and the spinocerebellar ataxias types 1, 2, 3, 6, 7, and 17. A transgenic mammal expressing at least 154 glutamine residues in a Sca1 or Sca7 locus has proven especially useful as an animal model for spinocerebellar ataxia type 1 and 7.

The present invention provides mammalian models of human polyglutamine repeat disease. The methods described herein provide high levels of expanded polyglutamine repeat disease protein expression, teach one of skill in the art the appropriate spatial and temporal distribution of expanded polyglutamine protein expression, provide the appropriate context for the generation of a knock-in model (i.e., provide teachings of the specific genes into which polyglutamine expansions are to be inserted to faithfully and accurately reproduce the disease phenotype), and further provide that the length of the polyglutamine repeat which is inserted into a target gene will all be crucial determining factors for disease pathogenesis, and for creating an appropriate model for polyglutamine disease.

More specifically, the present invention provides a knock-in mammal containing integrated into its genome a repeating polyglutamine sequence comprising at least 154 contiguous codons encoding glutamine and exhibiting at least one phenotype characteristic associated with a neurodegenerative disease.

In one embodiment, the knock-in mammal demonstrates one or more of the phenotype characteristics shown in Table 3.

In one embodiment, the knock-in mammal demonstrates accumulation of a protein comprising at least about 154 contiguous glutamine residues in neurons.

In one embodiment, the mammal demonstrates clinical symptoms of a disease selected from the group consisting of spinobulbar muscular atrophy (SBMA), Huntington's disease (HD), dentatorubral pallidoluysian atrophy (DRPLA), and the spinocerebellar ataxias types 1, 2, 3, 6,7, and 17.

In one embodiment, the mammal is a rodent.

In a further embodiment, the rodent is a mouse.

In one embodiment, the at least 154 codons comprise the codon CAG.

In a further embodiment, the contiguous codons encoding glutamine are present in a spinocerebellar ataxia gene.

In one embodiment, the spinocerebellar ataxia gene is selected from the group consisting of Sca1, Sca2, Sca3, Sca7, and Sca17.

In a further embodiment, the contiguous codons encoding glutamine are present in a genetic locus selected from the group consisting of Xq13-21, 4p16.3, 12p13.31, 6p23, 12q24.1, 14q32.1, 3p12-13, and 6q27.

In one aspect of the invention, the contiguous codons encoding glutamine are present in the coding sequence of a protein selected from the group consisting of an androgen receptor, huntingtin, atrophin-1, ataxin-1, ataxin-2, ataxin-3, ataxin-7, and a TATA-binding protein.

In a further aspect, the contiguous codons encoding glutamine are present in exon 8 of a Sca1 gene.

In one embodiment, the contiguous codons encoding glutamine are present in exon 3 of a Sca7 gene.

In one embodiment, the contiguous codons encoding glutamine are introduced into the genome of the mammal by a construct comprising a portion of a Sca1 exon 8.

In one embodiment, the contiguous codons encoding glutamine are introduced into the genome of the mammal by a construct comprising a portion of a Sca7 exon 3.

The present invention further provides a knock-in mammal containing integrated into its genome a repeating polyglutamine sequence comprising at least 154 contiguous codons encoding glutamine and exhibiting at least one phenotype characteristic associated with a neurodegenerative disease, and wherein the repeating polyglutamine sequence is integrated into a gene selected from the group consisting of a spinocerebelar ataxia gene, an androgen receptor gene, a Huntington's disease gene, and a DRPLA gene.

In one embodiment the spinocerebellar ataxia gene is selected from the group consisting of Sca1, Sca2, Sca3, Sca7, and Sca17.

In one embodiment, the spinocerebellar ataxia gene encodes a protein selected from the group consisting of ataxin-1, ataxin-2, ataxin-3, ataxin-7, and TATA-binding protein.

In a further embodiment, the contiguous codons are integrated into a chromosomal locus selected from the group consisting of Xq13-21, 4p16.3, 12p13.31, 6p23, 12q24.1, 14q32.1, 3p12-13, and 6q27

In one embodiment, the mammal demonstrates accumulation of a protein comprising at least about 154 glutamine residues in sequence in neurons.

In one embodiment, the mammal demonstrates clinical symptoms of a disease selected from the group consisting of the spinocerebellar ataxias types 1, 2, 3, 6, 7, and 17.

In one embodiment, the mammal demonstrates clinical symptoms of a disease selected from the group consisting of Huntington's disease, spinobulbar muscular atrophy, and dentatorubralpallidoluysian atrophy.

In one embodiment, the mammal is a rodent.

In a further embodiment, the rodent is a mouse.

In one embodiment, the at least 154 contiguous codons comprise the codon CAG.

The present invention also provides a knock-in mammal containing integrated into a chromosomal locus selected from the group consisting of Xq13-21, 4p16.3, 12p13.31, 6p23, 12q24.1, 14q32.1, 3p12-13, and 6q27, a repeating polyglutamine sequence comprising at least 154 contiguous codons encoding glutamine and exhibiting at least one phenotype characteristic associated with a neurodegenerative disease.

The invention still further provides a knock-in mammal containing integrated into a gene which encodes a protein selected from the group consisting of androgen receptor, huntingtin, atrophin-1, ataxin-1, ataxin-2, ataxin-3, ataxin-7, and TATA-binding protein, a repeating polyglutamine sequence comprising at least 154 contiguous codons encoding glutamine and exhibiting at least one phenotype characteristic associated with a neurodegenerative disease.

In one embodiment, the mammal demonstrates accumulation of a protein comprising at least about 154 glutamines in sequence in neurons.

In one embodiment, the mammal demonstrates clinical symptoms of a disease selected from the group consisting of spinobulbar muscular atrophy (SBMA), Huntington's disease (HD), dentatorubral pallidoluysian atrophy (DRPLA), and the spinocerebellar ataxias types 1, 2, 3, 6, 7, and 17.

In one embodiment, the mammal is a rodent.

In a further embodiment, the rodent is a mouse.

In one embodiment, the at least 154 contiguous codons comprise the codon CAG.

The invention still further provides a method for identifying an agent for treating a disease characterized by the abnormal presence of a polyglutamine sequence in a peptide or protein comprising the steps of: administering a candidate agent to a knock-in mammal having integrated into its genome a repeating polyglutamine sequence comprising at least 154 contiguous codons encoding glutamine and exhibiting at least one phenotype characteristic associated with a neurodegenerative disease; and monitoring the mammal to determine the effect of the agent on one or more phenotypic characteristic, wherein if the agent alters the at least one phenotype characteristic, then the agent is identified as an agent for treating a disease characterized by the abnormal presence of a polyglutamine sequence in a peptide or protein.

In one embodiment, the neurodegenerative disease is selected from the group consisting of spinobulbar muscular atrophy (SBMA), Huntington's disease (HD), dentatorubral pallidoluysian atrophy (DRPLA), and the spinocerebellar ataxias types 1, 2, 3, 6, 7, and 17.

In a further embodiment, the neurodegenerative disease is a spinocerebellar ataxia.

In one embodiment, the mammal demonstrates one or more of the phenotype characteristics shown in Table 3.

In one embodiment, the mammal demonstrates accumulation of a protein comprising at least 154 glutamines in sequence in neurons.

In one embodiment, the mammal is a rodent.

In a further embodiment, the rodent is a mouse

### BRIEF DESCRIPTION OF THE DRAWINGS

FIG. 1 demonstrates generation and expression of an expanded CAG allele at the Sca1 Locus. (A) Schematic representation of the targeting construct, the endogenous Sca1 allele, and the predicted structure of the mutant CAG expansion allele generated by a homologous recombination and a Cre-mediated excision event. (B) RT-PCR analysis using brain RNA from a 7-week-old Sca1^{154Q/2Q} knock-in mouse Sca1^{154Q/2Q} and a wild-type littermate (WT) with (+) or without (−) reverse transcriptase. The forward primer is located in exon 7 while the reverse primer sits right after the CAG repeat sequence in exon 8. The reaction gives 968 base pairs for the wild-type allele (arrow) and 1421 base pairs for the Sca^{154Q} allele (arrowhead). (C) Western blot analysis of whole brain extracts from a 7-week-old Sca1^{154Q/2Q} knock-in mouse (Ki/+) and a wild-type littermate (WT), probed with the 1C2 monoclonal antibody (left) and the 11750 antibody (right). Arrowhead and arrow indicate the mutant and wild-type ataxin-1 protein, respectively. The 1C2 antibody revealed non-specific cross reactant (*). Marker bands indicate 185, 119, and 85 kDa, respectively. (D) Western blot analysis of whole brain extracts (100 μg) from a 2-, 9-, and 25-week-old Sca1^{154Q/2Q} knock-in mouse using the 11750 antibody. Arrowhead and arrow indicate the mutant and wild-type ataxin-1 protein, respectively. (E) Western blot analysis of protein extracts (80 μg) from various parts of a 3-week-old Sca1^{154Q/2Q} mouse brain. Abbreviations are as follows: OB, olfactory bulb; BG, basal ganglia; HP, hippocampus; CX, cerebral cortex; PS, pons; CB, cerebellum; and SC, spinal cord. Arrowhead and arrow indicate the mutant and wild-type ataxin-1 protein, respectively. Relative densities of upper bands (=mutant ataxin-1) compared to lower bands (=wild-type protein) are as follows: OB, 0.29; BG, 0.09; HP, 0.21; CX, 0.09; PS, 0.29; CB, 0.58; and SC, 0.36. (F) Detection of aggregated ataxin-1 on immunoblot using the 11NQ antibody. Brain homogenates (150 μg) from a 2-, 20-, and 40-week old Sca1^{154Q/2Q} knock-in mouse Sca1^{154Q/2Q} and wild-type littermate (WT) were prepared in buffer containing urea/SDS and analyzed by SDS-PAGE.

FIG. 2 demonstrates the phenotype of Sca1^{154Q/2Q} mice. (A) Representative photographs of a 28-week-old Sca1^{154Q/2Q} mouse and its wild-type littermate. (B) Progressive weights of Sca1^{154Q/2Q} mice. Error bars indicate standard deviation. (C) Analysis of Sca1^{154Q/2Q} mice on the accelerating Rotarod apparatus. Performance of 5-week-old (left, n=8 in each group) and 7-week old (right, n=10 in each group) mice. Mice were trained four trials per day (a-d) for 5 days (D1-D5). Naive mutant mice and their wild-type littermates were tested in each test. Error bars indicate SEM. (D) Lifespan of Sca1^{54Q/2Q} mice. Percentage of Sca1^{154Q/2Q} knock-in (dashed line, n=10) and wild-type (black line, n=10) mice surviving at various ages is shown.

FIG. 3 demonstrates impaired learning and memory in Sca1^{154Q/2Q} mice. (A, B) Latency (A) and distance (B) to find the visible platform for Sca1^{154Q/2Q} and wild-type (WT) mice. (C) Swim speed in the visible platform task for Sca1^{154Q/2Q} and WT mice. (D) Latency to find the hidden platform in the Morris water task for Sca1^{154Q/2Q} and WT mice. (E, F) Probe trial data after hidden platform training in the Morris water task for Sca1^{154Q/2Q} mutant and WT mice. Quadrant search time (E) and number of platform crossings for the training quadrant are presented for the probe trials. (G, H) Pavlovian conditioned fear for Sca1^{154Q/2Q} and wild-type (WT) mice with percentage of intervals spent in freeze during the context test (F) and CS test (G). All data are expressed as the mean (±S.E.M.).

FIG. 4 demonstrates the effect of the knock-in mutation on hippocampal physiologic responses in area CA1. Hippocampal slices obtained from Sca1^{154Q/2Q} mice (E) or wild-type mice (J) were utilized in experiments to evaluate baseline synaptic function and short- and long-term forms of synaptic plasticity. Baseline synaptic transmission was determined by analyzing the slope of the population excitatory post synaptic potential (pEPSP) compared to the amplitude of the fiber volley. 5 and 8 week-old Sca1^{154Q/2Q} mutants show no significant differences in synaptic transmission (A, C). Paired-pulse facilitation (PPF) was unaffected in all age groups tested: 5, 8 and 24 week-old Sca1^{154Q/2Q} mutants (inset, A, C, E). Derangement of synaptic transmission was seen in 24 week-old Sca1^{154Q/2Q} mutants (E). Representative pEPSP traces (mean of 6 successive EPSPs) of baseline synaptic transmission for each age group are shown (inset, B, D, F) (Scale bars are 1 mV and 10 ms). Long-term potentiation (LTP) induced in stratum radiatum of area CA1 of the hippocampus is normal using 100 Hz stimulation (indicated by an arrow) in 5 week-old Sca1^{154Q/2Q} animals (B) and 8 week-old Sca1^{154Q/2Q} animals (D), however LTP was significantly reduced in 24 week-old mutants (90 min: WT 163±7.4, n=7 and Sca1^{154Q/2Q} 137±9.1, n=9, p=0.026) (F). Input-output curves were generated from 24 week-old Sca1^{154Q/2Q} knock-in mice by measuring the slope of the pEPSP at increasing stimulus intensities (G). (inset a) The stimulus intensity needed to elicit 50% of the maximum pEPSP slope for wild-type animals was determined to be 12.5 mV (G, a), compared to 17.5 mV needed to elicit a pEPSP with a similar slope for Sca1^{154Q/2Q} mice. With pEPSP slopes being equal, HFS-induced potentiation show a significant reduction in the amount of potentiation in Sca1^{154Q/2Q} mice (H). The pEPSPs immediately following HFS is significantly reduced in Sca1^{154Q/2Q} mutants (WT 2411±277.0, n=7 and Sca1^{154Q/2Q} 460±280.9, n=5, p=0.02) and is consistent for the duration of the experiment (90 min. post-tetanus: WT 1481±171.7, n=7 and Sca1^{154Q/2Q} 959±120.4, n=5, p=0.02). (Data points and error bars represent the mean±S.E.M.).

FIG. 5 characterizes nuclear inclusions (NIs) in Sca1^{154Q/2Q} brains. (A-K) Temporal and regional patterns of NI distribution. Cerebellar cortex (A, D, H), cerebral cortex (B, E, I), CA1 hippocampus (C, F, J), and anterior horn of spinal cord (G, K) stained with anti-ataxin-1 antibody 11NQ. (A-C) 5-week-old Scan1^{154Q/2Q} mouse. (D-G) 21-week old Sca1^{154Q/2Q} mouse. (H-K) 40-week-old Sca1^{154Q/2Q} mouse. Inclusions appear later in anterior horn neurons and Purkinje cells than in cortical neurons and hippocampal CA1 neurons. (L-O) CA1 hippocampal neurons of Sca1^{154Q/2Q} mice. NIs were revealed by anti-ataxin-1 antibody 11NQ (L) or anti-ubiquitin antibody (M), but not by anti-HDJ-2 antibody (N) in a 7-week-old mutant's brain. In a 40 week old mutant's brain, most of CA1 neurons possessed NIs stained with anti-HDJ-2 antibody (P). Insets show individual CA1 neurons with higher magnification. Original magnification for each photograph was ×200.

FIG. 6 depicts neurodegenerative and atrophic changes in Sca1^{154Q/2Q} brains. (A-D) Immunofluorescence confocal microscopy of 34 week old Sca1^{154Q/2Q} shows altered cerebellar morphology. Staining was performed with antisera against calbindin. (A, B) Sca1^{154Q/2Q} mouse. (C, D) wild-type mouse. Scale bars indicate 100 μm. (E) At 16 weeks, Sca1^{154Q/2Q} mice showed a statistically significant reduction in brain weight compared to wild-type littermates. Error bars indicate SEM. (F) Representative photographs of mid-sagittal sections from the mutant and wild-type brains (40-week-old). (G) Reduced calbindin immunofluorescence in 19-week-old Sca1^{154Q/2Q} mice. Calbindin immunofluorescence obtained from selected rectangular subsections of wild-type or the mutant cerebella was quantified and averaged (n=6, in each group).

FIG. 7A shows a simplified version of mouse Sca7 locus near exons 3 and 4. Targeting construct introduced 266 CAG repeats (inverted triangles) and flanking regions from human SCA7 into exon 3, obtaining a targeting frequency of 4%. Electroporation of Cre recombinase into the positive ES clones allowed the excision of the Neomycin(Neo)/Thymidine kinase (Tk) selection cassette (shown as an open box) from the targeted locus. P indicates a probe used for Southern analysis. Arrowheads indicate loxP sites. Abbreviations are as follows: 3, exon 3; 4, exon 4; 3^{e} engineered exon 3 with 266 CAG repeats; (B) Shows the results of southern analysis of EcoRI-digested tail DNA which revealed 15.2 kb wild-type and 10.3 kb mutant bands in Sca7^{266Q/5Q} mice; (C) Southern analysis showing that ataxin-7 is predominantly nuclear in the cerebellum and expanded ataxin-7 is expressed in vivo. N and C are nuclear and cytoplasmic, respectively; (D) Shows a picture of the gross morphology of Sca7^{266Q/5Q} mice; (E) Shows a plot of growth of the Sca7^{266Q/5Q} vs. wild-type mice.

FIG. 8 shows representative ERGs from wild-type and Sca7^{266Q/5Q} mice.

FIG. 9 shows retinal degeneration in Sca7^{266Q/5Q} mice Panel A shows the progressive downregulation of photoreceptor-specific genes in Sca7^{266Q/5Q} eyes. (B) shows the shortening of the outer segment in Sca7^{266Q/5Q} retina. At 10 weeks the outer segments in mutant mice become more dispersed than WT. Shortening continues as the mice age, and after 12 weeks, the outer segments are not longer detectable in Sca7^{266Q/5Q} retina. PE, pigment epithelium; OS, outer segment; IS, inner segment; ONL, outer nuclear layer; OPL, outer plexiform layer; INL, inner nuclear layer; IPL, inner plexiform layer; and GCL, ganglion cell layer.

FIG. 10 shows nuclear inclusions and apoptosis in Sca7^{266Q/5Q} retina. Panel A shows that NI distribution in the Sca7^{266Q/5Q} retina as detected by the 1261 antibody. Panel B shows the colocalization, using 1261 antibody and calbindin antibody, of mutant ataxin7 and NSE in cones of 10-week-old Sca7^{266Q/5Q} retina. Panel C shows that NIs colocalize with horizontal cells and amacrine cells in INL. Panel D shows that apoptosis of photoreceptors occurs after photoreceptor dysfunction. TUNEL positive signals were found in ONL. Panel E shows progressive activation of Müller glia in Sca7^{266Q/5Q} retina.

FIG. 11, panel A shows the results of rotarod analysis of Sca7^{266Q/5Q} mice vs. WT. Motor incoordination appears as early as 5 weeks in Sca7^{266Q/5Q} mice. Panel B shows a confocal micrograph of anti-calbindin labeled Purkinje cells of 16-week-old Sca7^{266Q/5Q} mice showing shrinkage of Purkinje cell bodies. Panel C shows a magnified view of the boxed portions of Panel B. Panel D shows the quantitation of cell body size in Sca7^{266Q/5Q} mice.

FIG. 12 shows the results of immunostaining of Sca7^{266Q/5Q} mouse cerebellum, indicating that mutant ataxin-7 gradually accumulates in the nuclei of cerebellar Purkinje cells in Sca7^{266Q/5Q} mice. Panel B shows a Northern analysis of the level of mutant Sca7 transcript in Sca7^{266Q/5Q} brain. Panel C shows a Western analysis of ataxin-7 expression in Sca7^{266Q/5Q} mice.

FIG. 13, panel A shows the results of immunohistochemical analysis of ataxin-7 protein expression in hippocampal cells. Panel B shows a measurement of baseline synaptic transmission in Sca7^{266Q/5Q} mice. Panel C shows an assessment of short term plasticity as measured by PPF. Panel D shows measurements of PTP in Sca7^{266Q/5Q} mice. Panel E shows the rescue of PTP in Sca7^{266Q/5Q} mice following successive trains of 100 Hz stimulation. Panel F shows the results of PTP analysis using a single train of 100 Hz stimulation in the presence of the NMDA receptor antagonists AP-5. Panel G shows a measurement of NMDA-receptor dependent LTP induced using one train of 200 Hz stimulation in the presence of AP-5. Panel H shows representative traces showing differences in baseline pEPSPs compared to pEPSPs during PTP.

### DETAILED DESCRIPTION OF THE INVENTION

The present invention provides an animal model for polyglutamine disease, and methods for screening a candidate agent for its ability to modulate a polyglutamine disease. The present invention is based, in part, on the discovery that certain neurodegenerative disease states are characterized by an expansion of a region of polyglutamine codons in certain genes, and that the disease state may be recapitulated in an animal model by utilizing a knock-in approach to insert at least 154 glutamine codons encoding at least 154 glutamine residues, preferably at least 160, 180, 200, 250, 270, 290, 300, 310, 330, 350, 400, 500, and up to 600 or more, into the coding region of the particular gene. More specifically, the invention provides an animal model of spinocerebellar ataxia (Sca) wherein the genes Sca1, Sca2, Sca3, Sca6, Sca7, or Sca17 are altered to comprise at least 154 glutamine codons in succession, thus resulting in at least one phenotype characteristic of the diseases SCA1, SCA2, SCA3, SCA6, SCA7, or SCA17. In addition, the invention provides an animal model of one or more of Huntington's disease, spinobulbar muscular atrophy (SBMA), and dentatorubralpallidoluysian atrophy (DRPLA), wherein the animal model comprises an expansion of at least 154 glutamine codons in succession in the huntingtin gene, the androgen receptor gene, or the atrophin-1 gene, respectively. The present invention also contemplates that the methods described herein may be useful to generate a knock-in mammalian model of other diseases such as psychiatric disease, and other diseases as yet undiscovered to result from polyglutamine expansion.

The present invention is also based, in part, on the discovery that the insertion of a polyglutamine repeat coding sequence into a polyglutamine disease gene, wherein the inserted repeat encodes a greater number of glutamine residues than is observed in the naturally occurring disease state, results in the onset of disease at an earlier time point than the naturally occurring disease. Accordingly, the present invention provides knock-in mammalian polyglutamine disease models which are advantageous over the other models taught in the art, in that the knock-in mammals taught herein present with a more robust disease phenotype which can be observed at a significantly earlier time point than models comprising a knock-in polyglutamine repeats having lengths similar to that observed in the naturally occurring disease state.

The present invention utilizes a “knock-in” approach to provide a transgenic mammal containing integrated into its genome a repeating nucleotide sequence encoding a polyglutamine comprising at least 154 codons encoding glutamine in succession. The present invention also features a method for testing the efficacy of an agent for treating a disease characterized by the abnormal presence of a polyglutamine sequence in a peptide or protein.

Definitions

A “knock-in” approach refers to the procedure of inserting the gene or the portion of a gene involved in the pathogenesis of a disease into the genome of a host. This may include, for instance, localizing the polynucleotide encoding a mutant polypeptide or protein to the locus encoding such polypeptide or protein or replacing an entire gene or coding region with a polynucleotide sufficient to encode a mutant polypeptide or protein. Accordingly, a “knock-in mammal” refers to a transgenic mammal produced using a “knock-in approach”.

A “transgenic” mammal is a mammal whose genome comprises a transgene or a portion of a transgene implicated in a disease process. The term therefore is meant to include mammals having a genetic insert or a genetic modification that encodes a mutant polypeptide or protein product implicated in causing, all or in part, a disease process. The transgene can be present in somatic cells or germ cells, or both. The transgenic mammal can be heterologous, homozygous, or hemizygous with respect to the transgene.

The term “homologous” refers to similarity of sequences (either protein or nucleic acid). Homologous polypeptides can contain stretches of sequences which are identical to a portion of the other. Alternatively, the sequences can still be homologous without containing stretches of sequences which are identical to a portion of the other. Sequences which are homologous can be determined by alignment against full length wild type polypeptides. Preferably, sequences which are homologous to each other are at least about 50% identical, more preferably at least about 60% identical, more preferably at least about 70% identical, more preferably at least about 80% identical, more preferably at least about 90% identical, and more preferably at least about 95% identical. Homologous sequences need not be of identical length.

“Phenotype” refers to a physical or behavioral manifestation indicative of a particular condition or disease. The term may include one or more physical symptoms indicative of or diagnostic for a disease process.

As used herein, “spinocerebellar ataxia gene” refers to a gene comprising a coding region comprising two or more glutamine codons in succession (referred to herein as the “polyglutamine region”), wherein expansion of the polyglutamine region to include at least double the number of glutamine codons compared to the wild type results in the occurrence of at least one phenotypic characteristic in the animal which is characteristic of spinocerebellar ataxia. Preferably, expansion of the polyglutamine region of a spinocerebellar ataxia gene will result in the primary phenotypic characteristics associated with spinocerebellar ataxia. As used herein, the primary phenotypic characteristics associated with spinocerebellar ataxia will vary depending on which specific spinocerebellar ataxia gene comprises a polyglutamine expansion, but generally, where the “spinocerebellar ataxia gene” is Sca1, a mammal comprising an expanded polyglutamine region within Sca1 will exhibit a phenotype including one or more of neurodegeneration (primarily reduced dendritic arborization of Purkinje neurons, and cell loss); motor incoordination; frank gait ataxia; memory and learning deficits; muscle atrophy; premature death; where the “spinocerebellar ataxia gene” is Sca2, a mammal comprising an expanded polyglutamine region within Sca2 will exhibit a phenotype including one or more of slow saccadic eye movement; occular hypo- or areflexia; cerebellar and brain stem Purkinje and granule cell degeneration; cytoplasmic inclusions of ataxin-2; where the “spinocerebellar ataxia gene” is Sca3, a mammal comprising an expanded polyglutamine region within Sca3 will exhibit a phenotype including one or more of neurodegeneration of the basal ganglia, brain stem, spinal cord, and dentate neurons of the cerebellum; progressive ataxia; neuronal intranuclear inclusions in pontine and dentate nuclei; reactive astrocytosis; where the “spinocerebellar ataxia gene” is Sca6, a mammal comprising an expanded polyglutamine region within Sca6 will exhibit a phenotype including one or more of slow, progressive ataxia; cerebellar atrophy; loss of cerebellar Purkinje cells; nuclear and cytoplasmic aggregation of polyglutamine expanded α1A calcium channels; where the “spinocerebellar ataxia gene” is Sca7, a mammal comprising an expanded polyglutamine region within Sca7 will exhibit a phenotype including one or more of ataxia; visual impairment; retinal degeneration; premature death; impared short-term potentiation; downregulation of photoreceptor genes; polyglutamine expanded ataxin-7 nuclear inclusions; and where the “spinocerebellar ataxia gene” is Sca17, a mammal comprising an expanded polyglutamine region within Sca17 will exhibit a phenotype including one or more of gait ataxia; nuclear inclusions and gliosis in caudate and putamen; diffuse cortical and cerebellar atrophy; cerebellar “torpedo bodies”.

As used herein, the term “candidate agent” means a biological or chemical compound such as a simple or complex organic or inorganic molecule, a peptide, a protein, an oligonucleotide, an antibody, an antibody derivative, or antibody fragment. A vast array of compounds can be synthesized, for example oligomers, such as oligopeptides and oligonucleotides, and synthetic organic compounds based on various core structures, and these are also included in the term “agent.” Also included in the term “agent” are antibodies which are generated in animals or synthesized recombinantly or by phage display. In addition, various natural sources can provide agents for screening, such as plant or animal extracts, and the like. Agents can be tested and/or used singly or in combination with one another.

As used herein, “monitoring” refers to any method of observing, measuring, documenting, or assessing a phenotype of a mammal. “Monitoring” of a mammal, preferably a knock-in mammal, may refer to continuous observation of at least one phenotype of the mammal, or alternatively, may refer to periodic observation of at least one phenotype of the mammal, or alternating observation of more than one phenotype of a mammal, or simultaneous observation of a plurality of phenotypes of a mammal. Monitoring may be performed using any of the phenotypic tests described herein, or which are known to those of skill in the art to be useful for measuring a phenotype which is associated with a neurodegenerative disease.

As used herein, “treatment” is an approach for obtaining beneficial or desired results, including and preferably clinical results. Treatment can involve optionally either ameliorating symptoms of the disease, preventing symptoms of the disease, or delaying progression of the disease. For purposes of this invention, beneficial or desired clinical results include, but are not limited to, amelioration or prevention of one or more of the following symptoms: motor incoordination, ataxia, cognitive deficits, muscle wasting, premature death, progressive Purkinje cell degeneration, and age related hippocampal synaptic dysfunction. Each of these symptoms is indicative of one or more of the following diseases: spinobulbar muscular atrophy (SBMA), Huntington's disease (HD), dentatorubral pallidoluysian atrophy (DRPLA), and the spinocerebellar ataxias types 1, 2, 3, 6, 7, and 17.

The terms “polynucleotide” and “nucleic acid”, used interchangeably herein, refer to a polymeric form of nucleotides of any length, either ribonucleotides or deoxyribonucleotides. These terms include a single-, double- or triple-stranded DNA, genomic DNA, cDNA, RNA, DNA-RNA hybrid, or a polymer comprising purine and pyrimidine bases, or other natural, chemically, biochemically modified, non-natural or derivatized nucleotide bases. It is understood that the double stranded polynucleotide sequences described herein also include modifications.

As used herein, “DNA” includes not only bases A, T, C, and G, but also includes any of their analogs or modified forms of these bases, such as methylated nucleotides, internucleotide modifications such as uncharged linkages and thioates, use of sugar analogs, and modified and/or alternative backbone structures, such as polyamides.

A polynucleotide is said to “encode” a polypeptide if, in its native state or when manipulated by methods well known to those skilled in the art, it can be transcribed and/or translated to produce the polypeptide or a fragment thereof. For purposes of this invention, and to avoid cumbersome referrals to complementary strands, the anti-sense (or complementary) strand of such a polynucleotide is also said to encode the sequence; that is, a polynucleotide sequence that “encodes” a polypeptide includes both the conventional coding strand and the complementary sequence (or strand).

Polyglutamine Repeat Sequences

The present invention provides knock-in mammals comprising an at least 154 codon expansion of a polyglutamine repeat region in one or more genes which are known to be linked to neurodegenerative disease. It is well known to those of skill in the art that a series of dominantly inherited, late-onset neurodegenerative disorders are caused by the expansion of a CAG trinucleotide repeat that encodes polyglutamine in the respective protein sequence (Watase et al., supra). Such polyglutamine expansion diseases include the spinocerebellar ataxias 1, 2, 3, 6, 7, and 17, Huntington's disease, spinobulbar muscular atrophy (SMBA) and dentatorubralpallidoluysian atrophy (DRPLA). There exists a need in the art, however, for an accurate animal model of one or more of these disease states which faithfully recapitulates the characteristics of the naturally occurring disease. The present invention is based, in part, on the discovery that the generation of a knock-in transgenic mammal, in which the polyglutamine repeat region of a particular gene is expanded to include at least 154 glutamine codons, results in the generation of an accurate model of the naturally occurring disease state. Accordingly, the polyglutamine expansion is preferably inserted into one or more of the genes which underlie the polyglutamine expansion disorders described above. In one embodiment, the polyglutamine expansion is present in one or more of the spinocerebellar ataxia genes. Preferably, the expansion is present in one or more of Sca1, Sca2, Sca3, Sca6, Sca7, or Sca17. The gene sequence for each of the Sca genes is known in the art, and, accordingly, following the description provided herein, one of skill in the art could readily generate a knock-in mammal based on the coding sequence for each of the Sca genes. Table 1 below provides the GenBank accession number for the mouse, human, and rat (where available) sequence for each of Sca1, Sca2, Sca3, Sca6, Sca7, or Sca17, and each of the proteins encoded thereby. The accession numbers and the sequences to which they refer are herein incorporated by reference.

TABLE 1

| Gene | Locus | Protein | Accession Number |
|---|---|---|---|
| Sca1 | 6p23 | Ataxin-1 | NM_009124 (mouse) |
| | | | NM_012726 (rat) |
| | | | NM_000332 (human) |
| Sca2 | 12q24.1 | Ataxin-2 | AF041472 (mouse) |
| | | | NM_002973 (human) |
| *Sca3 | 14q32.1 | Ataxin-3 | NM_004993 (human; variant 1) |
| | | | NM_030660 (human; variant 2) |
| Sca6 | 19p13 | α_{1A}-voltage- | NM_007578 (mouse) |
| | | gated calcium | NM_000068 (human; variant 1) |
| | | channel | NM_023035 (human; variant 2) |
| Sca7 | 3p12-13 | Ataxin-7 | NM_139227 (mouse) |
| | | | NM_000333 (human) |
| Sca17 | 6q27 | TATA-binding | BC016476 (mouse) |
| | | protein | NM_003194 (human) |

*mouse gene not yet identified

Using the accession numbers provided above, one of skill in the art could readily generate, using techniques described herein and which are known to those of skill in the art, a knock-in mammal in which the polyglutamine region of each of the above gene sequences is expanded to at least 154 glutamine codons (thus encoding a protein comprising at least 154 glutamine residues in succession). Alternatively, where the polyglutamine disease gene is Sca6 the polyglutamine region may be expanded to include at least 30 contiguous polyglutamine codons.

In a further embodiment, the invention comprises a knock-in mammal comprising a polyglutamine expansion in one or more genes that are known in the art to be implicated in Huntington's disease, SBMA, and DRPLA. In particular, the genes and encoded proteins that underlie these particular disease states are set forth in table 2 below. The accession numbers and the sequences to which they refer are herein incorporated by reference.

TABLE 2

| Gene | Locus | Protein | GenBank Accession Number |
|---|---|---|---|
| Androgen | Xq13-21 | Androgen | NM_013476 (mouse) |
| receptor | | receptor | NM_000044 (human) |
| gene | | | |
| HD | 4p16.3 | Huntingtin | XM_132009 (mouse) |
| | | | NM_002111(human) |
| DRPLA | 12p13.31 | Atrophin-1 | NM_007881 (mouse) |
| | | | BC051795 (human) |

The transgene (knock-in) of the present invention comprises a nucleic acid sequence encoding a polyglutamine sequence. The nucleic acid sequence may be repeating CAG codons. Due to the degeneracy of the genetic code, however, additional codons, for instance CAA, may substitute for one or more of the CAG codons so long as the substituting codon also encodes glutamine.

The present invention encompasses knock-in mammals having an abnormal polyglutamine expansion. The polyglutamine may be present in an otherwise wild type gene as shown above in tables 1 and 2, or it may be present in a gene encoding a functional analog, or allelic variant of one or more of the genes described above. A prediction of whether a particular polypeptide is a “functional analog” of another polypeptide can be based upon homology. Calculation of % homology therefore firstly requires the production of an optimal alignment, taking into consideration gap penalties. A suitable computer program for carrying out such an alignment is the GCG Wisconsin Bestfit package (University of Wisconsin, U.S.A.; Devereux et al., 1984, Nucleic Acids Research 12:387). Examples of other software that can perform sequence comparisons include, but are not limited to, the BLAST package (Ausubel et al., 1995, Short Protocols in Molecular Biology, 3rd Edition, John Wiley & Sons), FASTA (Atschul et al., 1990, J. Mol. Biol., 403-410) and the GENEWORKS suite of comparison tools. Both BLAST and FASTA are available for offline and online searching (Ausubel et al., 1999 supra, pages 7-58 to 7-60).

Although the final % homology can be measured in terms of identity, the alignment process itself is typically not based on an all-or-nothing pair comparison. Instead, a scaled similarity score matrix is generally used that assigns scores to each pairwise comparison based on chemical similarity or evolutionary distance. An example of such a matrix commonly used is the BLOSUM62 matrix—the default matrix for the BLAST suite of programs. GCG Wisconsin programs generally use either the public default values or a custom symbol comparison table if supplied. It is preferred to use the public default values for the GCG package, or in the case of other software, the default matrix, such as BLOSUM62.

Advantageously, the BLAST algorithm is employed, with parameters set to default values. The BLAST algorithm is described in detail on the world wide web at ncbi.nih.gov/BLAST/blast_help.html, which is incorporated herein by reference. The search parameters are defined as follows, and can be advantageously set to the defined default parameters.

Advantageously, “substantial identity” when assessed by BLAST equates to sequences which match with an EXPECT value of at least about 7, preferably at least about 9 and most preferably 10 or more. The default threshold for EXPECT in BLAST searching is usually 10.

BLAST (Basic Local Alignment Search Tool) is the heuristic search algorithm employed by the programs blastp, blastn, blastx, tblastn, and tblastx; these programs ascribe significance to their findings using the statistical methods of Karlin and Altschul (Karlin and Altschul 1990, Proc. Natl. Acad. Sci. USA 87:2264-68; Karlin and Altschul, 1993, Proc. Natl. Acad. Sci. USA 90:5873-7; see the world wide web at ncbi.nih.gov/BLAST/blast_help.html) with a few enhancements. The BLAST programs are tailored for sequence similarity searching, for example to identify homologues to a query sequence. For a discussion of basic issues in similarity searching of sequence databases, see Altschul et al (1994) Nature Genetics 6:119-129.

The five BLAST programs available at ncbi.nlm.nih.gov perform the following tasks: blastp—compares an amino acid query sequence against a protein sequence database; blastn—compares a nucleotide query sequence against a nucleotide sequence database; blastx—compares the six-frame conceptual translation products of a nucleotide query sequence (both strands) against a protein sequence database; tblastn—compares a protein query sequence against a nucleotide sequence database dynamically translated in all six reading frames (both strands); tblastx—compares the six-frame translations of a nucleotide query sequence against the six-frame translations of a nucleotide sequence database.

BLAST uses the following search parameters:

HISTOGRAM—Display a histogram of scores for each search; default is yes. (See parameter H in the BLAST Manual).

DESCRIPTIONS—Restricts the number of short descriptions of matching sequences reported to the number specified; default limit is 100 descriptions. (See parameter V in the manual page).

EXPECT—The statistical significance threshold for reporting matches against database sequences; the default value is 10, such that 10 matches are expected to be found merely by chance, according to the stochastic model of Karlin and Altschul (1990). If the statistical significance ascribed to a match is greater than the EXPECT threshold, the match will not be reported. Lower EXPECT thresholds are more stringent, leading to fewer chance matches being reported. Fractional values are acceptable. (See parameter E in the BLAST Manual).

CUTOFF—Cutoff score for reporting high-scoring segment pairs. The default value is calculated from the EXPECT value (see above). HSPs are reported for a database sequence only if the statistical significance ascribed to them is at least as high as would be ascribed to a lone HSP having a score equal to the CUTOFF value. Higher CUTOFF values are more stringent, leading to fewer chance matches being reported. (See parameter S in the BLAST Manual). Typically, significance thresholds can be more intuitively managed using EXPECT.

ALIGNMENTS—Restricts database sequences to the number specified for which high-scoring segment pairs (HSPs) are reported; the default limit is 50. If more database sequences than this happen to satisfy the statistical significance threshold for reporting (see EXPECT and CUTOFF below), only the matches ascribed the greatest statistical significance are reported. (See parameter B in the BLAST Manual).

MATRIX—Specify an alternate scoring matrix for BLASTP, BLASTX, TBLASTN and TBLASTX. The default matrix is BLOSUM62 (Henikoff & Henikoff, 1992). The valid alternative choices include: PAM40, PAM120, PAM250 and IDENTITY. No alternate scoring matrices are available for BLASTN; specifying the MATRIX directive in BLASTN requests returns an error response.

STRAND—Restrict a TBLASTN search to just the top or bottom strand of the database sequences; or restrict a BLASTN, BLASTX or TBLASTX search to just reading frames on the top or bottom strand of the query sequence.

FILTER—Mask off segments of the query sequence that have low compositional complexity, as determined by the SEG program of Wootton & Federhen (1993) Computers and Chemistry 17:149-163, or segments consisting of short-periodicity internal repeats, as determined by the XNU program of Claverie & States (1993) Computers and Chemistry 17:191-201, or, for BLASTN, by the DUST program of Tatusov and Lipman (see http://www.ncbi.nlm.nih.gov). Filtering can eliminate statistically significant but biologically uninteresting reports from the blast output (e.g., hits against common acidic-, basic- or proline-rich regions), leaving the more biologically interesting regions of the query sequence available for specific matching against database sequences.

Low complexity sequence found by a filter program is substituted using the letter “N” in nucleotide sequence (e.g., “NNNNNNNNNNNNN”) and the letter “X” in protein sequences (e.g., “XXXXXXXXX”).

Filtering is only applied to the query sequence (or its translation products), not to database sequences. Default filtering is DUST for BLASTN, SEG for other programs.

It is not unusual for nothing at all to be masked by SEG, XNU, or both, when applied to sequences in SWISS-PROT, so filtering should not be expected to always yield an effect. Furthermore, in some cases, sequences are masked in their entirety, indicating that the statistical significance of any matches reported against the unfiltered query sequence should be suspect.

NCBI-gi—Causes NCBI gi identifiers to be shown in the output, in addition to the accession and/or locus name.

Most preferably, sequence comparisons are conducted using the simple BLAST search algorithm provided on the world wide web at ncbi.nlm.nih.gov/BLAST. In some embodiments of the present invention, no gap penalties are used when determining sequence identity.

The nucleic acid of the transgene to be knocked-in to a mammal can be in the form of DNA, RNA, DNA analogs, RNA analogs, or a hybrid of DNA-RNA. The nucleic acid can also be single-stranded or double-stranded.

Vectors comprising the transgene are further provided by the present invention. Such vectors are useful for transforming or transfecting host cells. Vectors suitable for generating a knock-in mammal comprising at least 154 glutamine codons are known to those of skill in the art and are described further herein.

Preparation of Knock-In Mammals

Methods for generating gene constructs for use in generating knock-in mammals and the techniques for generating the mammals are known to those of skill in the art, and may be found, for example, in Sambrook et al., 1989, Molecular Cloning, A Laboratory Manual, 3^{rd} ed., Cold Spring Harbor Laboratory; Yoo et al., 2003, Neuron, 37: 383; Watase et al., 2002, Neuron, 34:905; Lorenzetti et al., 2000, Human Molecular Genetics, 9:779; and Lin et al., 2001, Human Molecular Genetics, 10:137.

Briefly, expanded polyglutamine codon regions (either CAG or CAA) of at least 154 polyglutamine codons (preferably about 160, 180, 200, 250, 270, 290, 300, 310, 330, 350, 400, 500, and up to 600 or more glutamine codons) are targeted into the specific genomic locus of a polyglutamine disease gene of interest. As used herein, a “polyglutamine disease gene” refers to a gene, which when comprising a polyglutamine expansion, is associated with a neurodegenerative disease. In a preferred embodiment, the expanded polyglutamine codon repeats are targeted into the genomic locus of one or more of the genes indicated in Table 1 or 2 above. In a particularly preferred embodiment, a knock-in mammal is produced which comprises a polyglutamine expansion in one or more of the Sca1, Sca2, Sca3, Sca6, Sca7, or Sca17 genes. As used herein, “targeted” means that the expanded glutamine repeat is inserted into a gene of interest in place of the endogenous polyglutamine repeat coding region present in the gene. Alternatively, “targeted” may refer to the insertion of an expanded glutamine repeat in a location of the gene which is distinct from the endogenous polyglutamine repeat coding region, or may alternatively refer to inserting an expanded glutamine repeat in a gene of interest such that it is contiguous with the endogenous polyglutamine repeat coding region. The expanded CAG or CAA tract is targeted to the locus using homologous recombination in embryonic stem cells (ES; Lorenzetti et al., supra). Methods for preparing the targeting construct have been described in the art and may be adapted from the methods described by Lorenzetti et al. (supra); Stacey et al., 1994, Mol. Cell. Biol., 14:1009; and Lin et al., 2001, Hum. Mol. Gen., 10:137. Chimeric mammals (e.g., chimeric mice) can then be generated by microinjection of at least two independent targeted ES clones into the blastocyst of a suitable mammal, such as the mouse strain C57BL/6J. The resulting chimeric mice are then mated either to wildtype animals of the same strain or a different strain, such as 129/SvEv female mice. Germline transmission of the expanded allele in the offspring from the matings can be confirmed using, for example, Southern blot analysis according to methods which are well known in the art. Alternatively, transmission of the expanded polyglutamine allele can be confirmed by other suitable methods known to those of skill in the art such as, but not limited to, PCR, Northern blot analysis, immunohistochemistry, or Western blot analysis. More specific methods for the generation of knock-in mammals comprising an expanded polyglutamine repeat region of at least 154 glutamine codons are described below in the Examples. It will be readily appreciated by one of skill in the art, that the methods described herein, coupled with the general skill in the art may be utilized to generate knock-in mammals of any type, comprising a polyglutamine expansion of at least 154 codons in a gene which is known to or suspected to be involved in a polyglutamine associated neurodegenerative disorder. It will also be appreciated by those of skill in the art that the methods of the present invention may be adapted to generate a knock-in mammal wherein the mammal is any suitable laboratory mammal such as a mouse, rat, guinea pig, and the like.

Disease Characteristics

The present invention provides a knock-in mammalian model of one or more polyglutamine repeat diseases, in which the expansion of the polyglutamine repeat region endogenously present in the disease gene is expanded to include at least 154 glutamine codons, and preferably at least 160, 180, 200, 250, 270, 290, 300, 310, 330, 350, 400, 500, and up to 600 or more glutamine codons. Accordingly, the present invention provides that the mammalian models described herein will recapitulate the phenotypic traits which are characteristic of one or more of the known polyglutamine repeat diseases known in the art and described further hereinbelow. As will be apparent to one of skill in the art, the particular phenotype observed in a knock-in mammalian polyglutamine repeat model as described herein will depend on the specific gene into which a polyglutamine expansion has been targeted. The polyglutamine disease phenotypes of the mammalian models of the present invention share generally several similar abnormalities including progressive neurodegeneration, neuronal dysfunction, neuronal loss, and aggregates of the polyglutamine expanded form of the polyglutamine disease protein (e.g., huntingtin, SCA1, CACNA1A, etc.) in the neuronal nucleus of affected brain regions.

Accordingly, the polyglutamine disease models of the present invention, depending on the specific genetic locus into which a polyglutamine expansion is inserted, will generally have one or more of the phenotypic characteristics described herein. As used herein, “phenotype characteristic associated with a neurodegenerative disease” refers to a physical or behavioral manifestation in a mammal which is established in the art as being at least one characteristic of a particular neurodegenerative disease, or alternatively, which is common to a plurality of neurodegenerative diseases. Table 3, below, shows “phenotype characteristics associated with a neurodegenerative disease” for each of the polyglutamine expansion diseases that may be modeled according to the methods of the present invention.

TABLE 3

| Polyglutamine | |
|---|---|
| Expansion | |
| Disease | Phenotypic characteristic |
| Huntington | motor task deficit; gait abnormality; ataxia; |
| Disease | clasping; seizures; reactive gliosis; striatal |
| | nuclear inclusions of huntingtin; weight loss; |
| | decreased striatal volume; overt |
| | neuropathology; Purkinje cell degeneration; |
| | behavior abnormalities; learning and memory |
| | defects; premature death |
| Spinobulbar | progressive loss of lower motor neurons; |
| Muscular | atrophyof anterior horn, bulbar region and |
| Atrophy | DRG; nuclear inclusions containing androgen |
| | receptor; gait abnormalities; muscle atrophy; |
| | gender-related motor impairment and |
| | neurodegeneration; premature death |
| SCA1 | neurodegeneration (including reduced dendritic |
| | arborization and heterotopia of Purkinje |
| | neurons, and cell loss); cytoplasmic vacuoles; |
| | nuclear inclusions of ataxin-1; motor |
| | incoordination; ataxia; memory and learning |
| | deficits; muscle atrophy; premature death |
| SCA2 | slow saccadic eye movement; occular hypo- or |
| | areflexia; cerebellar and brain stem Purkinje |
| | and granule cell degeneration; cytoplasmic |
| | inclusions of ataxin-2; nuclear inclusion of |
| | ataxin-2; premature death |
| SCA3 | neurodegeneration of the basal ganglia, brain |
| | stem, spinal cord, and dentate neurons of the |
| | cerebellum; Purkinje cell degeneration; |
| | progressive ataxia; nuclear inclusions in |
| | pontine and dentate nuclei; reactive |
| | astrocytosis; premature death |
| SCA6 | slow, progressive ataxia; cerebellar atrophy; |
| | loss of cerebellar Purkinje cells; nuclear and |
| | cytoplasmic aggregation of polyglutamine |
| | expanded α1A calcium channels; premature |
| | death |
| SCA7 | ataxia; visual impairment; retinal degeneration; |
| | premature death; impared short-term |
| | potentiation; downregulation of photoreceptor |
| | genes; polyglutamine expanded ataxin-7 |
| | nuclear inclusions; premature death |
| SCA17 | gait ataxia; nuclear inclusions and gliosis in |
| | caudate and putamen; diffuse cortical and |
| | cerebellar atrophy; cerebellar “torpedo bodies”; |
| | premature death |
| Dentato- | nuclear inclusions or atrophin-; neuronal loss |
| rubropallido- | in cerebral and cerebellar cortex, globus |
| luysian | pallidus, striatum, and the dentate (primary site |
| Atrophy | of neurodegeneration), subthalamic and red |
| | nuclei; premature death |

A phenotype characteristic associated with a neurodegenerative disease is generally abnormal compared to the same characteristic in a normal mammal (i.e., a mammal without a neurodegenerative disease). As used herein, a phenotype as measured in a knock-in mammal of the invention is said to be “abnormal” or to be an “abnormality”, and thus a “phenotype characteristic associated with a neurodegenerative disease”, wherein a measurement of the phenotype in the knock-in mammal is of a value which has a difference which is statistically significant (p≦0.05) when compared to the measurement of the same phenotype in a wild-type animal of the same species, age, and sex. Evaluation of a statistically significant difference by statistical tests known to those of skill in the art including, but not limited to Students t-test and ANOVA, between the treated and untreated mammals, whereby significance is granted to comparisons which generate a P value of at least≦0.05, preferably less than 0.049, but preferably between 0.049 and 0.001, but most preferably less than 0.001.

According to the present invention, in addition to the specific phenotypic characteristics described in table 3, the present invention provides animal models for polyglutamine disease wherein the onset of disease is accelerated in proportion with the size of the knock-in polyglutamine expansion. Thus, in addition to the presence of one or more of the above phenotypic characteristics, the invention provides that a phenotypic characteristic also includes the appearance of one or more of the above phenotypic characteristics at an earlier time than in a control animal. For example, the occurrence of cerebral atrophy is a phenomenon which generally occurs as part of the natural aging process. The present invention provides that such atrophy may be a phenotypic characteristic if it is observed to occur early in an animal's life, at a time prior to that when an aged animal would be expected to manifest the characteristic. In this way, the time of onset of a phenotypic characteristic may be used according to the present invention to identify an knock-in mammal of the invention as a useful model of a polyglutamine repeat disease.

Phenotype Assays

The present invention provides methods for generating knock-in mammalian models of polyglutamine repeat diseases. Accordingly, the models of the invention may be assessed for the presence of one or more phenotype characteristics which are characteristic of a neurodegenerative disease, preferably a polyglutamine disease. Assays which may be used to identify a phenotype characteristic are known to those of skill in the art, and include, but are not limited to the following.

Gait Analysis

For the analysis of a knock-in mammals gait to detect the presence of a gait abnormality, the feet or paws of a knock-in or control mammal may be painted, dyed, or otherwise colored prior to placing the mammal on a paper-lined runway, or other surface suitable for marking by the dye on the feet or paws of the mammal. The mammal is permitted to walk from one end of the runway to the other, thus leaving a record of their foot (or paw) placement on the paper. To obtain statistically relevant measurements of gait traits, measurements between footprints are made on several legible strides, wherein stride length is defines as the distance between adjacent prints made by the same foot or paw. Other useful measurements which may be made are base length, which is a measurement within each stride of one foot or paw to the opposite foot or paw (for a quadraped, this measurement may be made between opposite hind paws and opposite front paws). Overlap distance is a measurement of the distance in a quadraped, between the front and hind paws. To identify a phenotype characteristic in a knock-in mammal of the invention, gait measurements are compared between a knock-in mammal and a control mammal to identify an abnormality, wherein an abnormality is defined as a difference in measurement between the knock-in and control that shows statistical significance (e.g., p≦0.05).

Rotarod Analysis

To assess coordination and motor acquisition skills, a rotarod analysis test may be used. A test mammal (either a knock-in or control) is placed on an accelerating rod (e.g., type 7650; Ugo Basile, Milan Italy). The rod is accelerated from about 4 to about 40 rpm in about 5 minutes, although these parameters may be varied by one of skill in the art depending on the particular mammal to be studied. The time the mammal spends on the rod without falling is then recorded. In a typical testing paradigm, a mammal is placed on the rod in 2-4 trials for a period of 2-4 days, each trial lasting approximately 10 minutes. One of skill in the art will appreciate that the number and duration of trials may be varied depending on the particular mammal to be tested. Time to fall scores are then subjected to statistical analysis using, for example, ANOVA with repeated measurements. To identify a phenotype characteristic in a knock-in mammal of the invention, time to fall measurements are compared between a knock-in mammal and a control mammal to identify an abnormality, wherein an abnormality is defined as a difference in measurement between the knock-in and control that shows statistical significance (e.g., p≦0.05).

Learning and Memory Tests

Learning and memory of knock-in mammals of the invention may be assessed using methods which are well known to those of skill in the art including, but not limited to the visible platform test, Pavlovian conditioned fear test, and Morris water task.

The Pavlovian conditioned fear test may be conducted as described in the art (See, e.g., Paylor et al., 1994, Behav. Neurosci., 108, 810) using a Freeze Monitor system (San Diego Instruments). The test chamber is made of clear Plexiglas and surrounded by a photobeam deteciton system. The floor of the test chamber was a grid used to deliver an electric shock. The test chamber may be situated such that the external environment of the chamber, such as sound, light, temperature, etc. may be controlled. A mammal to be tested is placed in the test chamber and allowed to explore the chamber freely for two minutes. A white noise (e.g., 80 dB), which served as the conditioned stimulus (CS), was then presented for 30 sec followed by a mild (2 sec, 0.5 mA) foot-shock (the unconditioned stimulus, US). Either or both of the CS or US may be modified (e.g., increased or decreased in intensity depending on the particular mammal to be studied). Two min. later another CS-US pairing was presented. The mammal is then removed from the chamber 15-30 seconds later and returned to its starting environment. Freezing behavior should be recorded using the standard interval sampling procedure every 10 sec. Responses (run, jump, and vocalize) to the foot-shock are also recorded. Mammals that do not respond to the footshock should be excluded from analysis.

After a defined delay interval, the mammal is placed back into the test chamber for 5 min and freezing behavior is assessed every 10 sec (context test). One to two hours later, the mammal is tested for its freezing to the auditory CS. Environmental and contextual cues may be changed for the auditory CS test. For example,a black plexiglass triangular insert may be placed in the chamber to alter its shape and spatial cues, red house lights may replace the white house lights, the wire grid floor may be covered with black plexiglass, and odor cues, such as vanilla extract may be placed in the chamber to alter the smell. There are generally two phases during the auditory CS test. In the first phase (pre-CS), freezing is recorded for 3 min without the auditory CS. In the second phase, the auditory CS is turned on and freezing is recorded for another 3 min. The number of freezing intervals can be converted to a % freezing value. For the auditory CS test, the % freezing value obtained during the pre-CS period is subtracted from the % freezing value when the auditory CS was present. One of skill in the art will recognize that any or all of the above testing parameters may be altered depending on the particular mammal to be tested. To identify a phenotype characteristic in a knock-in mammal of the invention, fear response measurements are compared between a knock-in mammal and a control mammal to identify an abnormality, wherein an abnormality is defined as a difference in measurement between the knock-in and control that shows statistical significance (e.g., p≦0.05).

Behavioral phenotype characteristics may also be assessed using the Morris water task in which a test mammal attempts to locate a hidden escape platform in a circular pool of opaque water. Performance of the test mammal may be assessed using, for example a tracking system such as Noldus EthoVision (Leesburg, Va.). Each mammal to be tested may be given, for example, eight trials per day, in blocks of four trials for four consecutive days. The time taken to locate the escape platform (escape latency) and the distance traveled are determined. After trial 32 (assuming the above trial paradigm is used), each mammal is given a probe trial. During the probe trial, the platform is removed and each animal is allowed 60 sec to search the pool. The amount of time that each mammal spends in each quadrant is recorded (quadrant search time). The number of times a mammal crosses the exact location of the platform during training is also determined and compared with crossings of the equivalent location in each of the other quadrants (platform crossing). Selective search data in the probe trial is then analyzed by individual one-way (quadrants) repeated ANOVAs and Newman-Keuls post-hoc comparison tests. Two-way (genotype X gender) ANOVA may be used to compare the quadrant search time and platform crossing data for the training quadrant only between knock-in and wild-type or control mammals. To identify a phenotype characteristic in a knock-in mammal of the invention, search measurements are compared between a knock-in mammal and a control mammal to identify an abnormality, wherein an abnormality is defined as a difference in measurement between the knock-in and control that shows statistical significance (e.g., p≦0.05).

Mammals may be tested further on a visible platform test using the same proceedures described for the Morris water task, with the exception that a visual cue (such as a black cube attached to the top of the escape platform) is used. Generally the platform is located in the same place on each trial. Mammals are given, for example, eight trials a day, divided into two blocks of four trials each, on four consecutive days. Following the last trial, the platform is removed, and a 60 second probe trial is administered. Behavioral measurements are made similar to those described for the Morris water task and, accordingly, to identify a phenotype characteristic in a knock-in mammal of the invention, search measurements are compared between a knock-in mammal and a control mammal to identify an abnormality, wherein an abnormality is defined as a difference in measurement between the knock-in and control that shows statistical significance (e.g., p≦0.05).

Brain Slice Physiology

Assessment of phenotype characteristics in a knock-in mammal may be made from direct measurement of neuronal activity, recorded from physiologically maintained slices of the brain of a knock-in mammal.

To assess memory formation, the integrity of the hippocampal neuronal population may be assessed. Methods for performing electrophysiological analysis of hippocampal brain slices are known in the art, and may be adapted to permit analysis of any knock-in mammal of the invention. Briefly, hippocampal slices (400 μm) are prepared from the brains of a mammal to be tested and age-matched controls, as previously described (Roberson et al., (1996) J. Biol. Chem., 271, 30436-30441). One of skill in the art will appreciate that the exact thickness of the slice used according to the invention may vary depending on the particular mammal to be assayed. Slices are perfused (1 ml/min) with ACSF in an interface chamber maintained at 25° C. Field recordings of the Schaffer collateral synapse are monitored for a minimum of 10 minutes before recording to insure a stable baseline. Multiple traces may be averaged (e.g., 6 traces) to obtain an accurate measurement. Baseline stimulus intensities may determined from the stimulus intensity required to produce a population EPSP at 50% of the maximal response. LTP may be induced with two trains of 100 Hz stimulation for 1 second, separated by 20 seconds, with the identical stimulus intensity used for baseline recordings. LTP may be measured by methods known to those of skill in the art.

Cerebellar integrity may be determined by electrophysiological recording from cerebellar slices, according to methods known to those of skill in the art. Briefly, sagittal cerebellar slices of 200-250 μm thickness may be prepared from knock-in and/or control mammals as described previously (Aiba et al., (1994) Cell, 79, 377-388; Kano et al., (1997) Neuron, 18, 71-79). Whole-cell recording may be made from visually identified Purkinje cells using a 40× water immersion objective attached to an upright microscope (Olympus, BX-50WI) (Edwards et al., (1989) Pflugers Archiv., 414: 600-612). Resistance of patch pipettes should be approximately 3-6 MΩ when filled with an intracellular solution composed of (in mM): 60 CsCl, 10 Cs D-gluconate, 20 TEA-Cl, 20 BAPTA, 4 MgCl₂, 4 ATP, 0.4 GTP and 30 HEPES, (pH 7.3, adjusted with CsOH). The composition of standard bathing solution may be, for example, (in mM): 125 NaCl, 2.5 KCl, 2 CaCl₂, 1 MgSO₄, 1.25 NaH₂PO₄, 26 NaHCO₃ and 20 glucose, which was bubbled continuously with a mixture of 95% O₂ and 5% CO₂, although modifications to the particular ionic species and concentrations present in either the pipette or bath solution may be modified to specifically assess particular currents and/or channel properties. Bicuculline (10 μM) may be present in the saline to block spontaneous inhibitory postsynaptic currents. Ionic currents are recorded with a patch-clamp amplifier (Axopatch-ID, Axon Instruments). Stimulation and on-line data acquisition may be performed using the available software, such as PULSE software (HEKA, Germany). The signals are generally filtered at 3 kHz and digitized at 20 kHz. Fitting of the decay phases of measured EPSCs may be performed using the PULSE-FIT software (HEKA, Germany). In one embodiment, neuronal electrophysiological responses are recorded in response to stimulation of particular nerve populations in the cerebellum. For stimulation of climbing fibers and parallel fibers, a glass pipette with 5-10 μm tip diameter filled with standard saline may be used. Square pulses (duration, 0.1 ms; amplitude, 0-100V for climbing fiber stimulation, 1-10V for parallel fiber stimulation) may be applied for focal stimulation. Measurements may be made of membrane capacitance and/or EPSCs recorded in response to climbing fiber, or parallel fiber stimulation to determine the presence of a phenotype characteristic. To identify a phenotype characteristic in a knock-in mammal of the invention, electophysiological measurements (either LTP recorded from hippocampal slices, or EPSCs recorded from cerebellar Purkinje cells) are compared between a knock-in mammal and a control mammal to identify an abnormality, wherein an abnormality is defined as a difference in measurement between the knock-in and control that shows statistical significance (e.g., p≦0.05).

Immunohistochemical Analysis

In one embodiment, the invention provides that phenotype characteristics may be assessed by determining the presence or absence of nuclear or cytoplasmic inclusions of the mutant protein into which has been knocked-in a polyglutamine expansion of at least 154 glutamine codons. Immunohistochemical and immunofluroescence staining may be performed according to methods known in the art. To quantitate the amount of Purkinje cell dendritic arborization, sections of approximately 20 μm may be made from frozen cerebelli of control and knock-in mammals. Samples should be matched to prevent processing variations within each group. Sections may be stained with antibodies specific for the protein which contains the polyglutamine expansion (e.g., rabbit polyclonal anti-ataxin-1 (11NQ), anti-ataxin-7 antibody (1261; Yoo et al., 2003, Neuron, 37: 383), and sections may be analyzed to quantify the number of cytoplasmic or nuclear inclusions using image analysis software which is known in the art, such as NIH Image, or Neurolucida (Microbrightfield). To identify Purkinje cell neuropathology, sections may be labeled with anti-calbindin antibody to label all cytoplasmic regions of Purkinje cells. After staining with an antibody specific for the protein to be analyzed, sectoins are washed and mounted in Vectashield, and 0.5-1.0 μm optical sections are accumulated by confocal microscopy. The brightest 30 sections may be projected using the NIH Image J projection routine set for average intensity. From the resulting optical sections, a rectangular subsection of a cerebellar hemi-folium may be selected from the same region and same folium of each sample. The fluorescence intensity profile of this slab may be calculated using the plot profile routine of Image J and the resulting data sued to develop comparative fluorescence intensity and average change in intensity over the course of the cell dendritic arbor.

In addition, gross counts may be made of specific neuronal populations to determine cell death. The particular population which is counted will depend on the particular polyglutamine disease gene which is modified in the knock-in mammalian model. For example, where the gene is Sca1, cerebellar Purkinje cells may be counted, where the gene is a Huntington's disease gene, striatal neurons may be counted. Generally, both knock-in and control brains are sectioned and stained by an appropriate method, such as HE staining. Specific neuronal populations are counted in at least two adjacent sections from each brain under light microscopy, and statistical analysis may be carried out using ANOVA. To identify a phenotype characteristic in a knock-in mammal of the invention, cell counts are compared between a knock-in mammal and a control mammal to identify an abnormality, wherein an abnormality is defined as a difference in measurement between the knock-in and control that shows statistical significance (e.g., p≦0.05).

Methods of Screening for Biologically Active Agents

The knock-in, non-human mammals described herein are particularly useful for screening agents for biological activity, and in particular, for their potential efficacy in the treatment of a disease caused all or in part by an abnormal polyglutamine sequence in a polypeptide.

The methods of screening for biologically active agents generally feature exposing the knock-in mammal to an agent and determining the effect of the agent on the phenotype of the transgenic mammal. Detection of a decrease or increase in a characteristic of the phenotype is indicative of a biologically active agent. Accordingly, the methods of the present invention are based on the observation and measurement of a phenotypic characteristic in a knock-in mammal which is characteristic of a neurodegenerative disorder. A phenotype characteristic of a neurodegenerative disease refers to a physical or behavioral manifestation in a mammal which is established in the art as being at least one characteristic of a particular neurodegenerative disease, or alternatively, which is common to a plurality of neurodegenerative diseases as described hereinabove. Measurement of a phenotype associated with a neurodegenerative disease may be made by methods which are well known to those of skill in the art, and more specifically, the particular phenotypic test used to assess an characteristic will vary depending on the particular disease which a mammal is suspected of having. For example, where a polyglutamine expansion is targeted to the Sca1 gene, appropriate tests to monitor phenotypic characteristics include motor coordination and skill acquisition which may be measured by placing a mammal on an accelerating rotating rod, wherein the time the mammal is able to remain on the rod is a measurement of the mammal's motor skill and coordination, and is thus indicative of the presence or absence of an ataxia (Watase et al., 2002, Neuron, 34:905). Other tests and measurements which may be made to assess phenotypes associated with a neurodegenerative disease include the visible platform test, Pavlovian fear conditioning, gross ataxia test, the Morris water task, clasping, hippocampal slice physiology, cerebellar slice physiology, immunohistochemistry (e.g., to determine the presence or extent of nuclear inclusions comprising a polyglutamine disease protein, or other expression patterns of polyglutamine disease proteins such as, but not limited to an androgen receptor, huntingtin, atrophin-1, ataxin-1, ataxin-2, ataxin-3, α1A-voltage-gated calcium channel, ataxin-7, and TATA binding protein), immunofluorescence, and protein expression. Such tests are routine in the art for determining the presence of a phenotypic characteristic, and may be readily adapted to assess the phenotype of any given mammalian model. The above tests may be used, in conjunction with other suitable tests known to those of skill in the art, to determine a “phenotype measurement” which will be the output of the above measurements. For example, where the test is a rotating rod test, the phenotype measurement may be the time which a mammal is able to stay on the rod; where the test is a gross ataxia test, the phenotype measurement may be the stride distance or interval; where the test is the Morris water task, the phenotype measurement may be the time it takes a mammal to find the escape platform; one of skill in the art would readily be able to adapt one or more of the above tests to determine the presence of a phenotypic characteristic in a knock-in mammal of the invention. As used herein a phenotype, as measured by either the above tests or other tests known to those of skill in the art, is considered to be abnormal, or an abnormality if the phenotype measurement attains a statistically significant difference from the phenotype measurement of a control animal.

According to the present invention a phenotype characteristic associated with a neurodegenerative disease is said to be altered if the measurement of one or more of the characteristics is increased or decreased. That is, where a phenotype characteristic associated with a neurodegenerative disease is an abnormal phenotype (a phenotype which, when quantitiated by the methods of the invention is of a value which is different from the same phenotype measurement made in a control mammal, wherein the difference is statistically significant (p≦0.05)) the abnormal phenotype is said to be altered when the phenotype is either increased (made more abnormal) or decreased (made less abnormal and closer to the phenotype measured from a control mammal). According to the present invention, an abnormal phenotypic characteristic is considered to be “increased” where the particular characteristic becomes more severe (e.g., where the characteristic is premature death, the mammal dies earlier; where the characteristic is the presence of nuclear inclusions, the mammal has more nuclear inclusions per cell, or more cells with inclusions; where the characteristic is ataxia, the mammal has more severe ataxia; etc.), that is, there is a statistically significant (p≦0.05) difference in the measurement of the characteristic at a first reference point and the measurement of the more severe characteristic at a second reference point. According to the present invention, an abnormal phenotypic characteristic is considered to be “decreased” where the particular characteristic becomes less severe (e.g., where the characteristic is premature death, the mammal dies later; where the characteristic is the presence of nuclear inclusions, the mammal has fewer nuclear inclusions per cell, or fewer cells with inclusions; where the characteristic is ataxia, the mammal has less severe ataxia; etc.), that is, there is a statistically significant (p≦0.05) difference in the measurement of the characteristic at a first reference point and the measurement of the less severe characteristic a second reference point.

The methods described herein are applicable for testing an agent for efficacy in the treatment of a disease caused all or in part by an abnormal polyglutamine sequence in a polypeptide. Such methods feature exposing a knock-in non-human mammal to an agent, and determining whether there is an increase or decrease of the abnormal phenotype exhibited by the mammal following exposure to the agent. The increase or decrease of the abnormal phenotype may be assessed with any applicable behavioral test including those described herein. Such behavioral tests may be used to quantify such phenotypes as motor incoordination, ataxia and cognitive deficits. Routine microscopic techniques may be used to quantify such phenotypes as muscle wasting, gliosis, nuclear inclusions, and Purkinje cell degeneration. A decrease in the abnormal phenotype, as defined herein, indicates a substance useful for the treatment of disease. The agents may tested for their efficacy in treating any disorder caused all or in part by an abnormal polygluatamine such as spinocerebellar ataxia types 1, 2, 3, 6, 7, or 17, Huntington's disease, SBMA, or DRPLA.

In the context of the present invention, agents include but are not limited to a biological or chemical compound such as a simple or complex organic or inorganic molecule, a peptide, a protein, an oligonucleotide, a small molecule, an antibody, an antibody derivative, an antibody fragment, or a library of such chemical compounds, antibodies, antibody fragments, peptides or proteins.

Agents may be administered to the transgenic mammals systemically or locally. Suitable routes may include oral, rectal, or intestinal administration; parenteral delivery, including intramuscular, subcutaneous, intramedullary injections, as well as intrathecal, direct intraventricular, intravenous, intraperitoneal, intranasal, or intraocular injections, just to name a few. The response of the animals to the treatment may be monitored by assessing the reversal of disorders associated with neurodegenerative disorders. With regard to intervention, any treatments which reverse any aspect of neuronal degeneration should be considered as candidates for therapeutic intervention. However, treatments or regimens which reverse the constellation of pathologies associated with any of these disorders may be preferred. As used herein, “reverse” refers to a decrease in an abnormal phenotype such that the difference between the phenotype characteristic measured in the knock-in mammal compared to a control mammal is no longer statistically significant (p≦0.05). Dosages of test agents may be determined by deriving dose-response curves.

The transgenic animal model systems for polyglutamine disorders may also be used as test substrates in identifying environmental factors, drugs, pharmaceuticals, and chemicals which may exacerbate the progression of the neuropathologies that the transgenic animals exhibit.

In an alternate embodiment, the knock-in mammals of the invention may be used to derive a cell line which may be used as a test substrate in culture, to identify both agents that reduce and agents that enhance the pathologies. While primary cultures derived from the knock-in mammals of the invention may be utilized, the generation of continuous cell lines is preferred. For examples of techniques which may be used to derive a continuous cell line from the knock-in mammals, see Small et al., (1985) Mol. Cell Biol. 5:642-648.

As some neurons die while having a lower concentration of the protein than required to cause death of others, the knock-in mammals of the present invention may further be used to measure the vulnerability of particular neurons to the protein having a polyglutamine. Moreover, since the protein having a polyglutamine appears to be most soluble in neurons that are most susceptible to degeneration, differential solubility in different neurons may be used to predict the course of disease.

Human SCA1

In human SCA1 patients, expansions similar to those of the transgenic mammals of the present invention are associated with juvenile onset of clinical symptoms. One patient carrying an 82 CAG repeat developed initial symptoms at 4 years of age (Orr et al., (1993) Nature Genet., 4, 221-226), but the presence of neuropathological changes at this initial stage of the disease is still unknown. A larger amount of mutant ataxin-1 is required for mice to develop symptoms in their shorter life span. Alternatively, a longer polyglutamine tract within ataxin-1 induces neuronal degeneration at endogenous levels. Accordingly, the present invention provides knock-in mammalian models of human polyglutamine repeat disease which are useful in that they display an earlier onset of abnormal phenotypic characteristics, and the abnormalities present in response to endogenous levels of polyglutamine expanded protein expression. This, therefore, obviates the need to overexpress the expanded genes in the mammalian model.

### EXAMPLES

The following examples are provided to illustrate, but not limit, the present invention.

Knock-in Mammalian Model with Polyglutamine Expansion in Sca1

### Example 1

Generation of Sca1^{154Q/2Q} Mice and Analysis of Mutant Protein

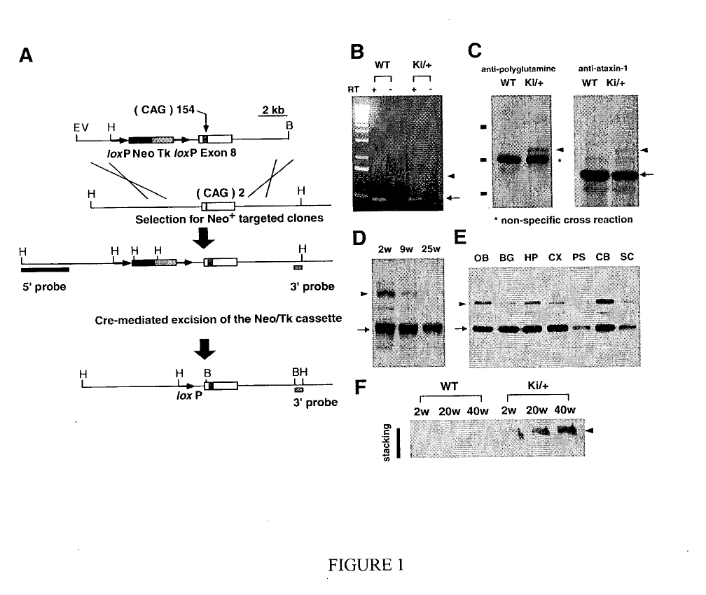

An expanded repeat of 154 CAGs was targeted into the mouse Sca1 locus (Sca1^{154Q}) as previously described (Lorenzetti, D., et al., (2000) Hum. Mol. Genet. 9, 779-785). Briefly, a targeting construct with 154 CAG repeats was prepared by culturing the SURE E. coli strain (Strategene) transfected with the bacterial plasmid vector previously made for generating Sca1^{78Q/2Q} mice. (Lorenzetti et al., (2000) Human Molecular Genetics 9(5), 779-785) A diagram of the construct is provided in FIG. 1. All Sca1 fragments used for the construct assembly were derived from a λ phage clone isolated from a 129/SvEv genomic library and subcloned in pBluescript KS+plasmid vector (Stratagene, La Jolla, Calif.). The mouse homolog of the SCA1 gene shares a high degree of homology with its human counterpart, but contains only two CAG units in its coding region (Banfi et al., (1996) Hum. Mol. Genet., 5, 33-40). A PCR-generated Msc1 HincII fragment was amplified from an SCA1 cDNA clone carrying a repeat of 154 CAG units and inserted into a BamHI Xhol fragment containing the 5′-most portion of the Sca1 coding region. Only two additional amino acid changes were introduced by this insertion. The entire exon 8 sequence was reconstituted by inserting this BamHI Xho1 clone into an XbaI ApaI genomic fragment spanning the intronic sequence at the 5′ and 3′ portions of the coding region. Subsequently, 4.7 kb. EcoRV BamHI and 3.2 kb ApaI BamHI genomic fragments spanning intronic sequences at the 5′ and 3′ ends, respectively, were ligated to the expanded exon 8. The selectable markers cassette was inserted into a KpnI site situated −0.8 kb from the 5′ intron-exon boundary. The entire exon 8 in the final construct was checked for sequence accuracy. Homologous recombination in ES cells inserted the chimeric Sca1 exon 8 with 154 CAG repeats and a cassette carrying the neomycin resistance gene (neo) and the thymidine kinase gene (Tk) flanked by two loxP sites (Abuin, et al., (1996) Mol. Cell. Biol., 16, 1851-1856) into the 5′ region of exon 8 (FIG. 1B). Correct targeting of the Sca1154Q allele in ES cells was confirmed by Southern blot analysis. To prevent potential interference of the selectable cassette with the transcription of the knock-in allele, the neo and Tk markers were removed from the targeted locus by Cre-mediated recombination between the flanking loxP sites (FIG. 1B) (Shellbourne, et al., (1999) Hum. Mol. Genet., 8, 763-774). A confirmed ES cell clone was injected into blastocysts to produce chimeric mice as described previously (Matilla, et al., (1998) Neurosci., 18, 5508-5516).

The obtained plasmids were checked for the entire exon 8 of the Sca1 gene by sequencing and then electroporated into ES cells derived from 129Sv/Ev strain (AB2.2). Homologous recombination was confirmed by Southern analysis as previously described (Lorenzetti et al., (2000), supra). Chimeric mice were crossed with C57B16J females. F1 and F2 animals are used for the studies. Both male and female F2 animals obtained from crosses between F1 heterozygous males and C57B16J wild-type females Crosses between F1 mice carrying an Sca1^{154Q} allele produced wild-type, heterozygous, and homozygous offspring in expected proportions.

Expression of the mutant transcript was assessed by RT-PCR using primers flanking the CAG repeat. Total RNA was extracted from a 7-week-old Sca1^{154Q/2Q} mouse brain. As shown in FIG. 1B, both mutant and wild-type transcripts produced almost equivalent band intensities, suggesting that mutant and wild-type alleles were similarly transcribed. The expression of mutant ataxin-1 was confirmed by Western analysis of mutant mouse brain extracts, which were prepared with 0.25M Tris-containing buffer (FIG. 1C). Surprisingly, in the adult mouse brain the 11750 antibody, which was raised against the C-terminal portion of ataxin-1 (Servadio et al., (1995) Nat. Genet., 10, 94-98), recognized mutant ataxin-1 as a much fainter ˜150 kDa band compared to its wild-type counterpart. This band also displayed immunoreactivity when the same blot was stripped and reprobed with 1C2 antibody, which detects long polyglutamine tracts. Neither 1C2, 11750, or the 11NQ antibody (which detects the N-terminal portion of ataxin-1) revealed any cleaved products derived from mutant ataxin-1.

To gain further insight into the apparent difference between levels of the wild-type and mutant protein in brain, proteins were extracted from mutant brains at three different ages and analyzed them by immunoblotting (FIG. 1D). Brain homogenates from each genotype were prepared by homogenizing either whole brain or specific sub-regions in 0.25 M Tris, pH 7.5, containing Complete (Roche) proteinase inhibitors. After homogenization, samples were briefly spun at 2500 rpm on the microcentrifuge (600 g) and supernatant was used for immunoblotting. For detection of aggregated forms of ataxin-1, samples were lysed in buffer containing 8 M urea, 4% SDS, 0.125 M Tris-HCl, pH 6.8, 12 mM EDTA, 3% β-mercaptoethanol, the proteinase inhibitor, and 0.002% bromophenol blue, incubated at 65° C. for 10 minutes and subjected to SDS-PAGE. Western blot analysis was performed as previously described (Skinner et al., (1997) Nature, 389, 971-974). Nitrocellulose blots were probed with polyclonal anti-ataxin-1 (11750VII, 1:3000, 11NQ, 1:3000) and monoclonal anti polyglutamine (1C2, 1:20000) antibodies.

Although mutant ataxin-1 created a distinct, strong band in a 2 week-old mouse brain, it became fainter as the mice got older and was barely detectable at 25 weeks of age. Nonetheless, immunohistochemical analysis with 11NQ revealed dense staining, often with intranuclear inclusion formation, in various neuronal populations from 12 week-old Sca1^{154Q/154Q} mouse brains. In order to analyze aggregated forms of mutant ataxin-1 on immunoblot, brain extracts were prepared for analysis with urea/SDS-containing buffer. Brain extracts from mutants but not control littermates gave distinct ataxin-1 immunoreactivity (ATX-1 IR) in the top part of the stacking gels (FIG. 1F). Densities of the ATX-1 IR got stronger as the mutants became older, indicating age-dependent increases in mutant ataxin-1 aggregation.

Since ataxin-1 is highly abundant in neurons, the expression and extractability of ataxin-1 was also compared among various brain regions of 3-week old mice. As shown in FIG. 1E, levels of wild-type ataxin-1 vary among brain sub-regions, with high levels in the cerebral cortex, basal ganglia, hippocampus and cerebellum. There are considerable differences in the extractability of mutant ataxin-1 in different brain regions. Mutant ataxin-1 gave fainter bands (i.e., was less extractable) in the basal ganglia and cerebral cortex than in the cerebellum, brain stem, spinal cord, and olfactory bulb.

### Example 2

Neurological Phenotype of Sca1154Q/2Q Mice

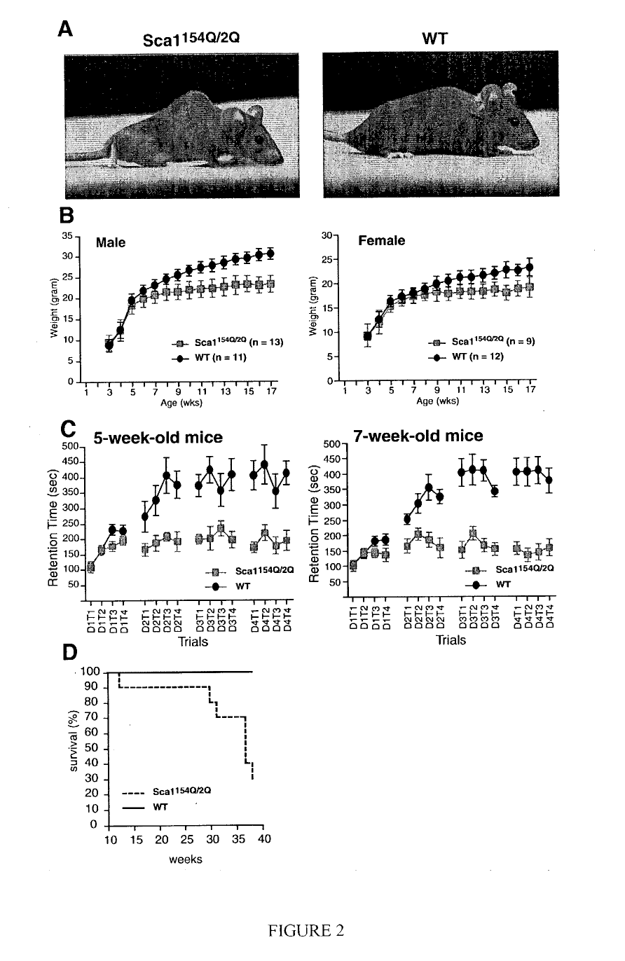

Up to 7 weeks of age, the mutant mice were indistinguishable from their wild-type littermates in home cage behavior. At 8 weeks of age, they began to show growth retardation. By 11 weeks of age, they weighed about 20% less than their wild-type littermates (FIG. 2B). The body weight of the Sca1^{154Q/2Q} mice peaked at around 20 weeks of age, after which the mice gradually lost weight as the disease progressed.

By 9 weeks, the mutants began demonstrating a clasping phenotype when suspended by the tail. The neurological phenotype progressed to a generalized muscle wasting, ataxia, and abnormal gait by 20 weeks of age. Severe kyphosis (cervical curvature of the spine) accompanied by atrophy of lower limb muscles was observed in the mutant animals at about 30 weeks (FIG. 2A). Premature death occurred between 35 and 45 weeks of age (FIG. 2D). None of the mutant animals survived past 50 weeks. The mice did not manifest any obvious seizures.

Naïve Sca1^{154Q/2Q} mice were tested on the accelerating rotarod apparatus at 5 and 7 weeks of age (FIG. 2C). An accelerated rotating rod test allowed the evaluation of coordination and motor skill acquisition (type 7650; Ugo Basile, Milan, Italy). Naïve F2 animals at 5 and 7 weeks of age were placed on the rod (30 cm diameter, 3 cm long) in four trials every day for a period of 4 days. Each trial lasted 10 minutes. The rod accelerated from 4 to 40 rpm in 5 minutes. The time the mice spent on the rod without falling was recorded. Behavioral scores were subjected to statistical analysis using ANOVA with repeated measures. Although the naïve Sca1^{154Q/2Q} mice had no overt gait ataxia at that time, their performance was significantly poorer than that of age-matched wild-type littermates at both 5 and 7 weeks of age (p<0.0005 and <0.0001, respectively).

### Example 3

Learning and Memory in Sca1^{154Q/2Q} Mice

Since cognitive impairment is one of the clinical features of SCA1, we assayed the cognitive function of Sca1^{154Q/2Q} mice. Spatial learning performance was assessed using the Morris water maze. Mice were trained in the Morris water task to locate a hidden escape platform in a circular pool (1.38 m diameter) of water. Mouse performance was recorded using a tracking system called Noldus EthoVision (Leesburg, Va.). Each mouse was given 8 trials per day, in blocks of four trials for four consecutive days. The time taken to locate the escape platform (escape latency) and the distance traveled were determined. After trial 32, each animal was given a probe trial. During the probe trial, the platform was removed and each animal was allowed 60 sec to search the pool. The amount of time that each animal spent in each quadrant was recorded (quadrant search time). The number of times a subject crossed the exact location of the platform during training was determined and compared with crossings of the equivalent location in each of the other quadrants (platform crossing). Selective search data in the probe trial were analyzed by individual one-way (quadrants) repeated ANOVAs and Newman-Keuls post-hoc comparison tests. Two-way (genotype X gender) ANOVA was used to compare the quadrant search time and platform crossing data for the training quadrant only between mutant and wild-type mice.

Since Sca1^{154Q/2Q} mutants have notable motor impairment, we tested a group of Sca1^{154Q/2Q} mice at 7-8 weeks of age on a visible platform task in which the animals are required to find a slightly submerged fixed platform whose location is marked by a large proximal cue. Mice were trained on the visible platform test using the same procedures as those described for the hidden-platform test as described above, with the exception that a black cube (7.5×7.5 cm) was attached to the top of the escape platform. The bottom of the block was 10 cm above the surface of the platform. The platform was located in the same place on each trial. Mice were given eight trials a day, divided into two blocks of four trials each, on 4 consecutive days. Following the last trial the platform was removed and a 60-sec probe trial was administered.

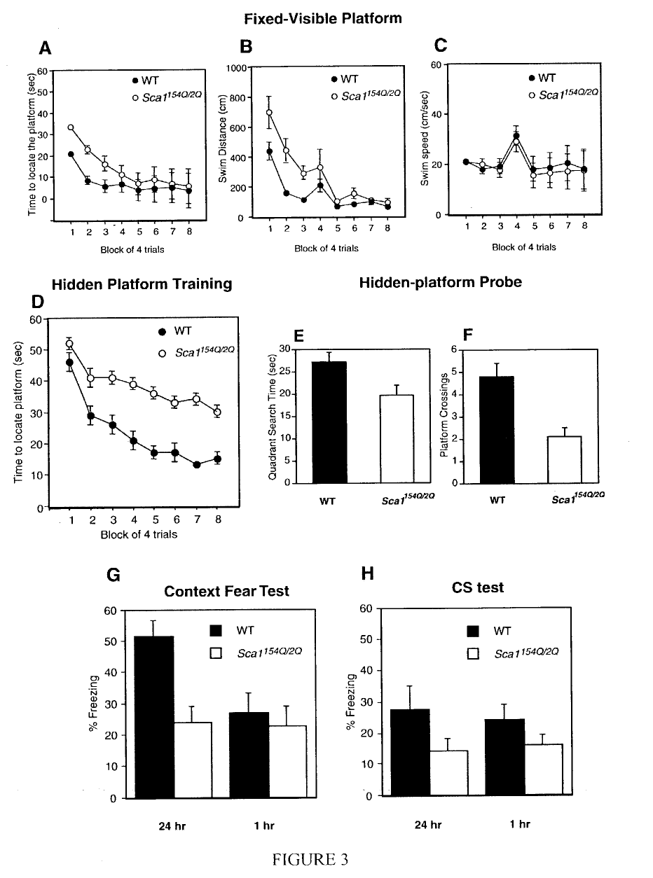

Overall the Sca1^{154Q/2Q} mutants took considerably longer to locate the platform than wild-type mice (p=0.000001) (FIG. 3A), but a genotype×trial interaction (p=0.000002) indicates that the performance of the two genotypes was dependent on the trial number. Sca1^{154Q/2Q} mutants took more time and swam a greater distance to find the platform during the early trials (i.e., trial blocks 1-6, p<0.05), but performed as well as wild-type mice on trial blocks 7 and 8 (p=0.21) (FIGS. 3A, B). There was no overall difference (p=0.564) in swim speed between Sca1^{154Q/2Q} and wild-type mice (FIG. 3C), suggesting that simple motor impairment was not interfering with the performance of the mutant mice on this test.

The hidden platform test (Morris water maze) measures the ability of an animal to learn the spatial relationship between extramaze cues and the platform. Performance is assessed by recording the time it takes to locate the platform during training and characterizing the search pattern during a probe trial that is given after training. Sca1^{154Q/2Q} mutant mice took significantly more time (FIG. 3D) to locate the hidden platform than wild-type mice (p=0.0000004). FIGS. 3E and F show that during the probe trial, wild-type mice spent significantly more time in the training quadrant than the Sca1^{154Q/2Q} mutant mice (p=0.025), and wild-type mice crossed the exact place where the platform had been located more often than Sca1^{154Q/2Q} mutant mice (p=0.001). These data indicate that Sca1^{154Q/2Q} mice have impaired spatial learning performance

The conditioned fear test assays Pavlovian learning and memory. Mice were 6-8 weeks old at the start of testing. Performance in a conditioned fear paradigm was measured as described before (Paylor et al., (1994) Behav. Neurosci., 108, 810-817) using a Freeze Monitor system (San Diego Instruments). The test chamber was made of clear Plexiglas and surrounded by a photobeam detection system. The floor of the test chamber was a grid used to deliver an electric shock. The test chamber was placed inside a sound-attenuated chamber (Med Associates); in the front of the chamber are windows through which the mice can be observed. A mouse was placed in the test chamber (house lights “ON”) and allowed to explore freely for two minutes. A white noise (80 dB), which served as the conditioned stimulus (CS), was then presented for 30 sec followed by a mild (2 sec, 0.5 mA) foot-shock (the unconditioned stimulus, US). Two min. later another CS-US pairing was presented. The mouse was removed from the chamber 15-30 seconds later and returned to its home cage. Freezing behavior was recorded using the standard interval sampling procedure every 10 sec. Responses (run, jump, and vocalize) to the foot-shock were recorded. Animals that did not respond to the footshock were excluded from analysis.

After a defined delay interval, the mouse was placed back into the test chamber for 5 min and freezing behavior was assessed every 10 sec (context test). One to two hours later, the mouse was tested for its freezing to the auditory C. Environmental and contextual cues were changed for the auditory CS test. A black plexiglass triangular insert was placed in the chamber to alter its shape and spatial cues, red house lights replaced the white house lights, the wire grid floor was covered with black plexiglass, and vanilla extract was placed in the chamber to alter the smell. Finally, the sound attenuated chamber was illuminated with red house lights. There were two phases during the auditory CS test. In the first phase (pre-CS), freezing was recorded for 3 min without the auditory CS. In the second phase, the auditory CS was turned on and freezing was recorded for another 3 min. The number of freezing intervals was converted to a % freezing value. For the auditory CS test, the % freezing value obtained during the pre-CS period was subtracted from the % freezing value when the auditory CS was present. For the present study, Sca1^{154Q/2Q} mutants and wild-type mice were trained and then tested either 1 hour or 24 hours later. The results from the Context test (FIG. 3G) show that there was a significant genotype×delay interval interaction (p=0.0436). Sca1_{154Q/2Q} mice displayed significantly less freezing than wild-type mice during the Context test after a 24 hour delay (p=0.026), but not after a 1 hour delay (p=0.883). In contrast to the findings for the Context test, there was no genotype×delay interval interaction for the conditioned stimulus (p=0.687) (FIG. 3H). Thus the difference between Sca1^{154Q/2Q} and wild-type mice depends on the type of test (Context vs. CS) and the delay interval (24 hour vs. 1 hour).

Mice with hippocampal dysfunction have been shown to have impaired context-based, but not cued, conditioned fear (Frankland et al., (1998) Behav. Neurosci., 112, 863-74; Logue et al., (1997) Behav. Neurosci., 111, 104-113; Jiang et al., (1998) Neuron, 21, 799-811) and impaired spatial learning in the hidden-platform version of the Morris water task (Logue et al., (1997) Behav. Neurosci., 111, 104-113). The pattern of poor performance for the Sca1_{154Q/2Q} mutants on the learning and memory tasks is consistent with altered hippocampal function

### Example 4

Hippocampal Synaptic Function in Sca1^{154Q/2Q} Mice

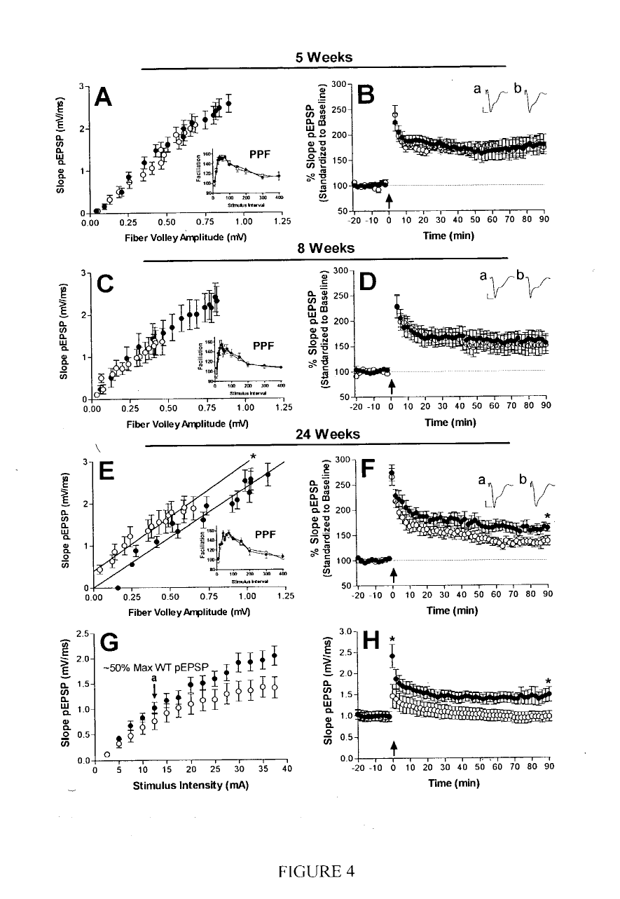

It was previously demonstrated that ataxin-I is required for normal short-term synaptic plasticity in hippocampal CA1 neurons; ataxin-1 knock-out mice displayed impaired paired pulse facilitation (Matilla et al., (1998) J. Neurosci, 18, 5508-5516). The effect of the mutation on the synaptic function of CA1 neurons was therefore examined using extracellular recordings from hippocampal slices. Hippocampal slices (400 μm) were prepared from either 5, 8 or 24 week-old Sca1^{154Q/2Q} mice and age-matched controls, as previously described (Roberson et al., (1996) J. Biol. Chem., 271, 30436-30441). Slices were perfused (1 ml/min) with ACSF in an interface chamber maintained at 25° C. Field recordings of the Schaffer collateral synapse were monitored for a minimum of 10 minutes before recording to insure a stable baseline. Responses are presented as an average of 6 individual traces. Baseline stimulus intensities were determined from the stimulus intensity required to produce a population EPSP at 50% of the maximal response. LTP was induced with two trains of 100 Hz stimulation for 1 second, separated by 20 seconds. (indicated by an arrow, FIGS. 4B, D, F, H), with the identical stimulus intensity used for baseline recordings.

Hippocampal slices from 5-week-old animals were tested. No significant difference between 5-week-old Sca1^{154Q/2Q} mice and wild-type controls was observed in baseline synaptic transmission, paired pulse facilitation (PPF), or long-term potentiation (LTP) (FIGS. 4A and B).

A hallmark of polyglutamine disease is the appearance of neuronal intranuclear inclusions (NIs). No NIs were observed in 5-week old mouse brains, but ubiquitinated NIs were abundant in CA1 hippocampal neurons at 7 weeks of age. The electrophysiological responses of CA1 neurons at 8 weeks of age were therefore examined. Mutant animals had normal input-output functions, PPF, and LTP (FIGS. 4C and D), suggesting that NIs do not alter synaptic transmission or plasticity at this age. Older (24 week-old) Sca1^{154Q/2Q} mice, however, exhibited a decrease in the input-output functions for stimulation of Schaffer collateral inputs into area CA1 (FIG. 4E, p<0.02), suggesting that basal synaptic transmission is impaired in the older Sca1^{154Q/2Q} mutant hippocampus. The derangement in synaptic transmission manifested itself as an alteration in the pEPSP relative to fiber volley amplitude (FIG. 4E). In addition, mutant slices exhibited a decrease in maximum EPSP response (FIGS. 4E, 4G).

Although derangement in baseline synaptic transmission may complicate evaluation, LTP analysis was conducted in the 24 week-old Sca1^{154Q/2Q} mutants. Tetanic stimulation in slices from the 24 week-old animals elicited normal post-tetanic potentiation (PTP), but a reduced potentiation is seen immediately following high-frequency stimulation (HFS). This diminution of potentiation lasted 90 minutes post-tetanus (FIG. 4F).

The LTP-induction protocol uses a stimulus intensity that elicits ˜50% of the maximum pEPSP to evaluate both baseline synaptic transmission and the period of LTP-inducing, 100 Hz high frequency stimulation. As shown in FIG. 4G, the maximum pEPSP slope in 24 week old mutants is considerably lower than that of the wild-type controls. In other words, beginning with a lower absolute EPSP magnitude, the Sca1^{154Q/2Q} mutants achieve 50% of the maximum slope of the pEPSP and thus achieve a lower EPSP magnitude during LTP-inducing high-frequency stimulation. This could underlie the difference in Sca1^{154Q/2Q} LTP induction, since the EPSP magnitude during stimulation could have an effect on the overall potentiation produced. Therefore, in the next set of experiments the stimulus intensity required to elicit 50% of the maximum pEPSP in our wild-type group (FIG. 4G, arrow at α) was determined and then adjusted the stimulus used in our Sca1^{154Q/2Q} mutants to give a pEPSP with a similar EPSP slope (see baseline in FIG. 4H). It was found that the stimulus intensity required to give approximately equal pEPSPs (˜950 μV/ms) was 12.5 mA and 17.5 mA for wild-type and Sca1^{154Q/2Q} mutants, respectively. At the stated stimulus intensities, the recorded baseline pEPSPs are indistinguishable for both animal groups prior to HFS (FIG. 4H).

Even when absolute EPSP magnitude was normalized between control and the knock-in animals, there was a diminution of LTP in the mutant animals (FIG. 4H, p=0.02). These results suggest that the hippocampal LTP deficit observed in the 24 week-old Sca1^{154Q/2Q} mice reflects a deficit in HFS-dependent synaptic plasticity.

Sca1^{154Q/2Q} knock-in animals exhibited an age-dependent derangement of hippocampal synaptic physiology. Five- and 8-week-old animals displayed normal baseline synaptic transmission, normal PPF, and normal LTP, but 24-week-old animals exhibited two alterations in baseline synaptic function. First, there was a significant leftward shift of the fiber volley-EPSP relationship for baseline synaptic transmission indicating altered stimulus-response function for synaptic transmission in the hippocampal area. The second change that was observed was a decrease in the maximum pEPSP slope in the mutant animals along with an apparent attenuation of LTP. LTP was diminished even when baseline transmission was normalized to 50% of maximum and stimulus intensity was normalized so that both wild-type and knock-in animals evoked the same absolute EPSP slope. Hence, there is altered LTP in Sca1^{154Q/2Q} animals, with the important caveat that these apparent changes are superimposed on an altered baseline synaptic response.

### Example 5

Electrophysiology of Purkinje Cells in Young Sca1^{154Q/2Q} Mice

Sca1^{154Q/2Q} mice developed motor incoordination at 5 weeks of age. At this stage the Purkinje cells appeared grossly normal by calbindin immunofluorescence, though we could not rule out subtle changes in distal dendrites. To probe the mechanism underlying impaired motor coordination in young mutant animals, the electrophysiological properties of cerebellar Purkinje neurons at a relatively early age (6 to 11 weeks) were examined using whole-cell recordings in cerebellar slices.

Sagittal cerebellar slices of 200-250 μm thickness were prepared from the wild-type (P45-79, n=4) and Sca1^{154Q/2Q} (P46-78, n=4) mice as described previously (Aiba et al., (1994) Cell, 79, 377-388; Kano et al., (1997) Neuron, 18, 71-79). Whole-cell recording was made from visually identified Purkinje cells using a 40× water immersion objective attached to an upright microscope (Olympus, BX-50WI) (Edwards et al., (1989) Pflugers Archiv., 414: 600-612). Resistance of patch pipettes was 3-6 MΩ when filled with an intracellular solution composed of (in mM): 60 CsCl, 10 Cs D-gluconate, 20 TEA-Cl, 20 BAPTA, 4 MgCl₂, 4 ATP, 0.4 GTP and 30 HEPES, (pH 7.3, adjusted with CsOH). The composition of standard bathing solution was (in mM): 125 NaCl, 2.5 KCl, 2 CaCl₂, 1 MgSO₄, 1.25 NaH₂PO₄, 26 NaHCO₃ and 20 glucose, which was bubbled continuously with a mixture of 95% O₂ and 5% CO₂. Bicuculline (10 μM) was always present in the saline to block spontaneous inhibitory postsynaptic currents. Ionic currents were recorded with a patch-clamp amplifier (Axopatch-1D, Axon Instruments). Stimulation and on-line data acquisition were performed using the PULSE software (HEKA, Germany). The signals were filtered at 3 kHz and digitized at 20 kHz. Fitting of the decay phases of EPSCs was done with the PULSE-FIT software (HEKA, Germany). For stimulation of climbing fibers and parallel fibers, a glass pipette with 5-10 μm tip diameter filled with standard saline was used. Square pulses (duration, 0.1 ms; amplitude, 0-100V for climbing fiber stimulation, 1-10V for parallel fiber stimulation) were applied for focal stimulation.

We first compared passive membrane properties of Purkinje cells by recording membrane currents in response to hyperpolarizing voltage steps from the holding potential of −70 to −80 mV. As reported previously (Llano et al., (1991) J. Physiol (Lond), 434, 183-213), the decay of the current was biphasic, and could be described by the sum of two exponentials. From their time constants, we calculated several parameters representing passive properties of Purkinje cells based on the model equivalent circuit of PCs described by Llano et al. (1991) (Table 4). This model distinguishes two regions in the Purkinje cell. Region 1 represents the soma and the main proximal dendrite; region 2 represents the main part of dendritic tree. The lumped membrane capacitance of regions 1 and 2 were calculated as C1 and C2, respectively. It was found that the lumped membrane capacitance of the dendritic tree (C2) was significantly smaller in Sca1^{154Q/2Q} mice than wild-type mice (p≦0.01, t test) (Table 4). Given the discernible reduction of dendritic arbor at advanced stages of disease, these results likely reflect a reduction in the average total membrane area of the dendritic tree in young Sca1^{154Q/2Q} mice.

TABLE 4
Electrophysiological Parameters of the Purkinje Cell

| | Wild-Type | Sca1^{154Q/2Q} |
|---|---|---|
| Passive membrane properties | n = 14 | n = 16 |
| C1 (pF) | 184 ± 19 | 164 ± 16 |
| C2 (pF) | 1038 ± 60** | 776 ± 50** |
| R1 (MΩ) | 6.8 ± 0.3 | 6.6 ± 0.3 |
| R2 (MΩ) | 7.4 ± 0.5 | 8.40 ± 0.6 |
| R (MΩ) | 219.8 + 22.0 | 408.4 ± 97.7 |
| CF-EPSC | n = 24 | n = 24 |
| 10%-90% rise time (ms) | 0.4 ± 0.01 | 0.4 ± 0.01 |
| ^{b}Decay time constant (ms) | 5.4 ± 0.20 | 5.0 ± 0.24 |
| ^{c}Chord conductance (nS) | 111.6 ± 7.4 | 100.3 ± 11.4 |
| PF-EPSC | n = 10 | n = 8 |
| 10%-90% rise time (ms) | 1.0 ± 0.04 | 1.0 ± 0.13 |
| ^{b}Decay time constant (ms) | 8.9 ± 0.93 | 8.5 ± 0.92 |
| ^{a}Parameters for passive membrane properties were calculated according to the model described by Liano et al. (1991), which distinguishes two regions of Purkinje cells: region 1, which represents the soma and the main proximal dendrites, and region 2, which represents the dendritic tree. C1 and C2 represent the lumped membrane capacitance of regions 1 and 2, respectively. R1 represents the pipette access resistance. Region 2 is linked to region 1 by resistor R2, which represents | | |
| #the lumped resistance of the dendritic tree of PCs. The values of C1, C2, R1, and R2 were calculated from the initial capacitive currents in response to hyperpolarizing voltage steps (500 ms duration) from −70 to −80 mV. The value of R3 was measured from the steady state currents in response to hyperpolarizing voltage steps (500 ms duration) from −80 to −85. | | |

^{b}Decay time constants for CF- and PF-EPSCs were measured by fitting the EPSC decay with a single exponential. Holding potential was −80 mV.
^{c}Chord conductance was measured from CF-EPSCs recorded at the holding potentials of −20 mV and +40 mV.
All data are expressed as mean ± SEM.
**p <0.01 (t test)

The kinetics and short-term plasticity of climbing fiber-mediated (CF) and parallel fiber-mediated (PF) EPSCs were assessed. The decay time constant was obtained by fitting the decay phases of the EPSCs with single exponentials. No significant difference between those young Sca1^{154Q/2Q} mice and wild-type controls were observed in the kinetics of the rise and decay times for both the CF- and PF-EPSCs (Table 4), paired-pulse facilitation of PF-EPSC, or in paired-pulse depression of CF-EPSC. The degree of multiple climbing fiber innervation of Purkinje cells was similar between wild-type mice and Sca1^{154Q/2Q} mice. In addition, there was no significant difference between the two mouse strains in the chord conductance for CF-EPSCs (Table 4). These results suggest that synapse formation and function at this age are not altered in the mutant animals.

### Example 6

Nuclear Inclusions in Different Brain Regions

Immunohistochemical analysis of ataxin-1 in control adult mouse brains shows that ataxin-1 localizes predominantly to the nuclei of neurons throughout the nervous system. Immunohistochemical and immunofluorescence staining were performed as previously described elsewhere (Skinner et al., (1997) Nature, 389, 971-974; Cummings et al., (1999) Neuron, 24, 879-892). The following antisera were used to stain brain tissue: rabbit polyclonal anti-ataxin-1 (11NQ), monoclonal anti-HDJ2/HSDJ (1:200, Neomarkers), monoclonal anti-ubiquitin (1:200, Novo Castra), monoclonal anti-calbindin (1:1000, Sigma). For the quantitation of dendritic arborization, sections 20 or more microns thick were taken from frozen fixed cerebelli of control and Sca1^{154Q/2Q} mice at 19 weeks of age. Samples were matched to minimize processing variations within each group. Sections were stained with anti-calbindin antibody which labels all cytoplasmic regions of Purkinje cells. After washing and mounting in Vectashield, 37 0.5 micron optical sections were accumulated with a Biorad 1024 confocal microscope using the same parameters for each sample. The brightest continuous series of 30 sections was projected using the NIH Image J projection routine set for average intensity. From the resulting 15 micron optical slabs, a rectangular subsection of a cerebellar hemi-folium was selected from the same region and same folium of each sample. The fluorescence intensity profile of this slab was calculated using the plot profile routine of Image J and the resulting data used to develop comparative fluorescence intensity and average change in intensity over the course of the cell dendritic arbor.

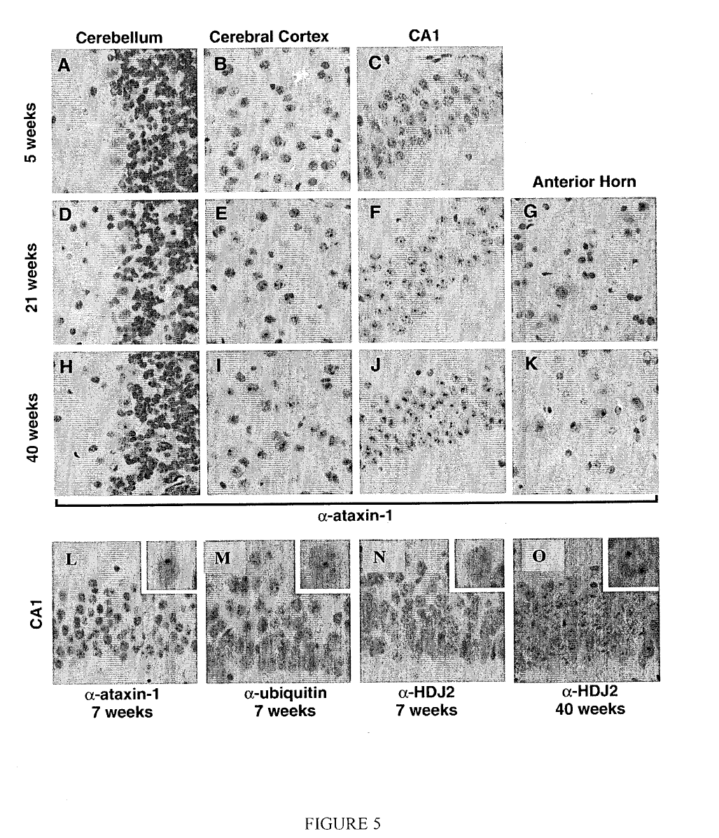

By light microscopic analysis, NIs were first evident as a single small dot at 6 weeks of age in cortical neurons, CA1 hippocampal neurons, and thalamic nuclei. In end-stage brains, large NIs with dense staining were observed in these neurons. In addition, inclusions within neurons were observed in various other regions (caudate, putamen, cerebellum, brain stem, and spinal cord). Mapping of NIs in the mutant brains revealed considerable differences between neuronal groups in the appearance and abundance of NIs. For example, neither hypothalamic or cerebellar granule neurons develop NIs. However, ataxin-1 is expressed at very low levels in these neurons. Since it is known that, in SCA1, marked neurodegeneration occurs in specific areas (Purkinje cells, red nucleus, olivary complex, and motor neurons in the anterior horn), NI formation was examined in the analogous mouse neurons to examine the relationship between NIs and neurodegeneration. Interestingly, NIs were detected less frequently in these most susceptible neuronal groups than in cortical or hippocampal pyramidal neurons, which show only mild neurodegeneration in SCA1 patient tissue (FIG. 5). Less than 0.5% of Purkinje cells harbored tiny NIs before 20 weeks of age, while more than 80% of the neurons in the cerebral cortex and hippocampus contained NIs at the same age.

NIs in the knock-in mice stained positive for ubiquitin (FIG. 5M), as they are in B05 mouse and human tissue. Antibodies to HDJ2 chaperone failed to reveal any NIs in 7 week-old-brain, merely diffusely staining the cytoplasm (FIG. 5N). As the NIs grow over time, some of NIs in the mutant hippocampus, cortex, and inferior olive become positive for HDJ2 by 18 weeks of age. In mutant brains from 40 weeks of age, most of the NIs in those areas (but not those in Purkinje cells) were recognized by the antibodies. Hsc70, which was also detected in ataxin-1 nuclear aggregates in Purkinje cells of B05 mice, was observed in most hippocampal or cortical NIs in 40-week old, but not 7 week-old, mutant brains. These results suggest that the involvement of HDJ2 chaperone as well as Hsc70 in NI may be secondary to the initial formation of NI or sequestration of ubiquitin. Alternatively, during the initial phase of NI formation, these chaperone proteins may be in a complex in the NI that might allow only limited access to antibodies.

In several other polyglutamine diseases, CREB binding protein (CBP) has been found to be sequestered into NIs, and interference of CBP-mediated transcription may be involved in neuronal toxicity (McCampbell et al, (2000) Hum. Mol. Genet., 9, 2197-2202; Nucifora et al, (2001) Science, 291, 2423-2428). Brain sections derived from 7-, 20-, and 40-week-old Sca1^{154Q/2Q} mice were stained with two antibodies, A-22 and C-20 (Santa Cruz, Calif.), that recognize either the amino- or carboxyl-terminus of the protein. Although the CBP-IR was largely restricted to the nucleus, none of the ataxin-1 NIs were stained with them.

The tissue distribution of NIs in Sca1^{154Q/2Q} mice is consistent with previous reports that NI formation does not initiate disease in SCA1 transgenic animals (Klement et al., (1998) Cell, 95, 41-53). NIs were rare and appeared only very late in the course of the disease in the mutant Purkinje neurons and spinal cord neurons, where the worst neurodegenerative pathology was apparent. On the other hand, NIs already populated most neurons of the cerebral cortex, CA1 hippocampus, and thalamus by the time mice reached 21 weeks of age. This is reminiscent of the neuropathology of a juvenile SCA1 patient in whom NI formation was detected in pontine neurons and some cortical neurons but not in other areas, such as remaining Purkinje neurons. In a study of tissue from patients with Huntington's disease, Kuemmerle et al. also showed that inclusions form more frequently in non-affected neuronal populations (Kuemmerle et al., (1999) Ann. Neurol., 46, 842-849).

There are both temporal and regional alterations in mutant ataxin-1 extractability, which declines as the animals get older. The densities of the mutant and wild-type ataxin-1 were unequal even in 2-week-old brain extracts. It is noteworthy that the extractability of mutant ataxin-1 was higher in the cerebellum than the cerebral cortex and basal ganglia. In the cerebellum, ataxin-1 is expressed in various types of neurons, but immunfluorescence analysis indicated that its expression in granule neurons is much lower than that in Purkinje neurons.

An obvious pattern emerges from consideration of the earliest behavioral manifestations (impaired motor coordination), degrees of protein extractability (greatest in cerebellar tissue), electrophysiological changes (early, in Purkinje cells), and sites of neurodegeneration (most notable in Purkinje cells) and NI formation (late, in Purkinje cells): neurons that sequester ataxin-1 into NIs only late in the course of the disease are also the cells that are most severely affected by polyglutamine toxicity. Ataxin-1 toxicity was much greater when it was not sequestered in NIs (Cummings et al., (1999) Neuron, 24, 879-892). NIs appear in vulnerable neurons at an early stage in SCA1 transgenic mice due to the very high expression of the mutant protein (˜50× endogenous levels). Their relative absence in the most vulnerable neurons in human patients as well as the Sca1^{154Q/2Q} mice suggests that the selective neuropathology in polyglutamine diseases reflects a complex combination of mutant protein expression levels, protein solubility, and the presence of factors that enable neurons to sequester ataxin-1 into NIs and thereby curb its toxicity.

This observation is supported by recent findings on the pathophysiology of Alzheimer's disease (AD) and of Parkinson's disease (PD). A hallmark of pathology in both disorders is the presence of hard-to-solubilize, ubiquitin-positive deposits (senile plaques and Lewy bodies, respectively). But it is the soluble, not insoluble, pool of Aβ that correlates with neurodegeneration in AD (McLean et al., (1999) Ann. Neurol., 46, 860-866). The situation is similar in PD: mutations of parkin cause early-onset autosomal recessive PD characterized by the absence of Lewy bodies (Shimura et al., (2001) Science, 293, 263-269). Mutant parkin may lead to the accumulation of a soluble, non-ubiquitinated form of α-synuclein in parkin-deficient PD brains, accelerating neuronal loss and causing the earlier disease onset.

### Example 7

Neuropathology of Sca1^{154Q/2Q} Mice

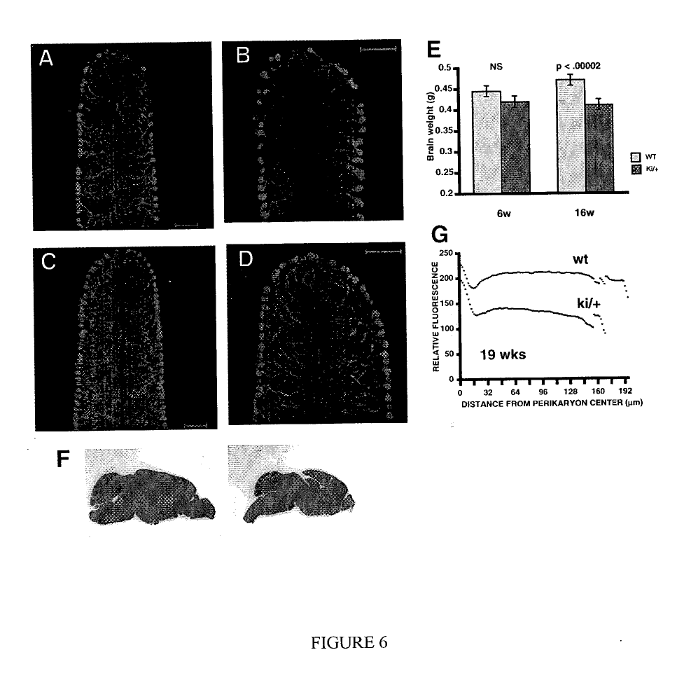

Several mutant and wild-type mouse brains were examined from different age groups, ranging from 4 weeks juvenile) to 42 weeks (end-stage). Juvenile mutant mice showed no gross abnormalities, but brains from end-stage mutants were consistently smaller than those from littermate controls. 16-week-old mutants had significantly reduced brain weight (p<0.00002 by ANOVA, FIG. 6E). Brain sections obtained from the aged mice (40 weeks) showed uniform atrophy, with dilatation of all the ventricles (FIG. 6F).

Inspection with anti-calbindin immunofluorescence uncovered no gross pathological changes in Purkinje cells from 17 week old Sca1^{154Q/2Q} mice. Using quantitative analysis, however, on cerebellar slices from the 19-week-old animals, wild-type Purkinje cells had dendritic arbors were found with approximately 50% higher fluorescence than their mutant counterparts (FIG. 6G). Similar alterations were noted on slices from a pair of 9-week-old animals (data not shown). The lower fluorescence in mutant Purkinje cells indicates the paucity of fine dendritic arbor.

By 34 weeks of age, dendritic arborization was obviously reduced in cerebella from mutant animals (FIGS. 6A-6D) and Purkinje cell loss was noticeable. Cell loss among Purkinje neurons and hippocampal pyramidal neurons was quantified on mid-saggital sections stained with haematoxylin and eosin (HE) in 40 week-old mutants and their littermates. There were significantly (p<0.01) fewer Purkinje neurons in the mutant cerebella (wild-type; 255.3±9.4, Sca1^{154Q/2Q} mice; 196.8±11.6, mean±SEM, per section; n=4 animals). The relative intact population of hippocampal pyramidal neurons in the mutant brains (wild-type; 497.3±12.3, Sca1^{154Q/2Q} mice; 461.7±34.2, mean±SEM, per section; n=3 animals) underscores the greater vulnerablility of Purkinje neurons to mutant ataxin-1 toxicity. Purkinje cell loss did not appear to result from apoptotic mechanisms, based on negative Tunel staining. Reactive astrocytosis was detected in the spinal cord by glial fibrillary acidic protein (GFAP) immunoreactivity; staining was most intense in the dorsal columns, but enhanced immunoreactivity was also observed in the ventral part, including the anterior horn. There was no increase in GFAP staining in other brain regions such as the cerebellum and brain stem.

Reduced dendritic arborization of Purkinje neurons was noted early in the course of disease in the knock-in mice. Purkinje cell loss (-20%) did not occur until the end-stage of disease. Other neuronal groups such as the hippocampal neurons suffered dysfunction while not revealing notable cell loss. This correlates with human data since neuronal dysfunction goes on for many years before cell loss is a significant problem. In humans, cell loss is most prominent in Purkinje cells (the first neurons to become impaired), whereas hippocampal and cortical neuron loss is minimal, despite the fact that these patients suffer cognitive deficits (Zoghbi,et al., (1995) Semin. Cell Biol., 6, 29-35).

Knock-In Mammalian Model with Polyglutamine Expansion in Sca7

### Example 9

Generation of Sca7^{266Q/5Q} Mice

The present invention provides a knock-in mammalian model of neurodegenerative disease, having a polyglutamine expansion of at least 154 glutamine codons. Preferably the expansion is present in one or more of the genes Sca1, Sca2, Sca3, Sca6, Sca7, the huntingtin gene, the androgen receptor gene, or the atrophin-1 gene. In one embodiment of the invention, the polyglutamine is present in the mouse Sca7 gene, and is thus useful to generate a knock-in mouse model of spinocerebellar ataxia type-7.

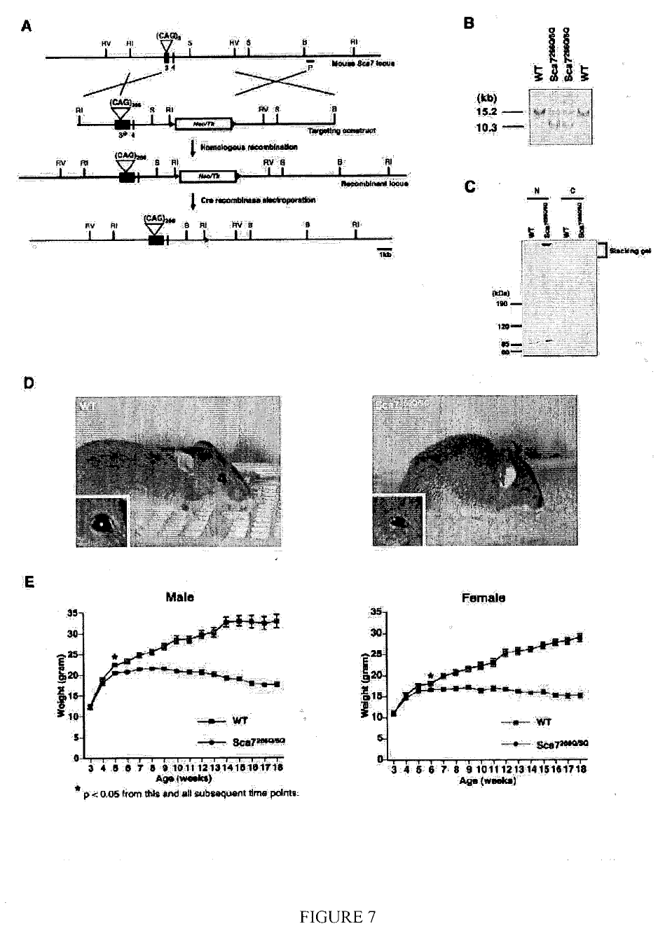

Mouse Sca7 is highly homologous to human SCA7, with the peptide sequence showing 88.7% identity (Ström et al., 2002, Gene, 285:91). Whereas the CAG repeat trace in human SCA7 is polymorphic (ten repeats are the most common), mouse Sca7 has only five CAG repeats in the corresponding region. 266 CAG repeats were introduced into the mouse Sca7 locus using homologous recombination in embryonic stem (ES) cells (FIG. 7A). Briefly, the 129/SvEv mouse genomic DNA library was screened using mouse EST clone W10624 (GenBank Accession number), which is homologous to the 5′ end sequence of human SCA7 exon 3. Two clones (named 6A and 17A) were pulled out from this screen and were used to construct a targeting vector. hSCA7.forward (5′-TTGTAGGAGCGGAAAGAATGTC-3′) and hSCA7.reverse (5′-ATCACTTCAGGACTGGGCAGAG-3′) were used to PCR amplify 306 CAG repeats and flanking regions from the published patient sample (Benton et al., 1998). 100 ng of patient genomic DNA was used in a 50 μl reaction tube containing 50 μM of each primer, 12.5% dimethylsulfoxide, 250 μM each dNTP, 1.5 mM MgCl₂, 50 mM KCI, 10 mM Tris-HCI (pH 8.3), and 4 U of Taq DNA polymerase. Cycling conditions were as follows: 95° C. for 5 min followed by 35 cycles of denaturing at 94° C. for 1 min, annealing at 62° C. for 1 min, extension at 72° C. for 2 min, and final extension at 72° C. for 7 min. 198 bp Notl/HindIII fragment of mouse Sca7 exon 3 was subcloned into pZErO 2.0 (Invitrogen). In order to introduce Apal site in position 153, this clone was PCR amplified with M13 reverse primer and mSca7-Apal primer (5′-GACGGCGGGCCCGGGGACACCACACCTCG-3′). This changed 5′-GGCACC-3 into 5′-GGGCCC-3′ in position 150-155 of mouse Sca7. PCR product from the human patient sample was double digested with Notl and Bsp120I (isoschizomer of Apal) and ligated into the mouse Sca7 exon 3 with new Apal site. Several subsequent ligation steps were followed to generate the Sca7 knock-in construct (19.8 kb). A selectable cassette containing the Neomycin resistance gene (Neo) and the Thymidine kinase gene (Tk) flanked by two IoxP sites was inserted into the Sac1 site, which is about 2.3 kb downstream from exon 4. The 306 CAG repeats were highly unstable and contracted to 266 during the multiple ligation steps. Sca7 knockin construct with 266 CAG repeats were electroporated into embryonic stem (ES) cells, and subsequent electroporation of Cre recombinase into the positive ES clones allowed the excision of Neomycin (Neo)/Thymidine kinase (Tk) selection cassette. Correctly targeted ES cells were injected into C57BL/6J blastocysts to generate chimeras, which were crossed to C57BL/6J females to obtain Sca7^{266Q/5Q} mice. Germline transmission of a targeted allele was confirmed using both tail DNA Southern analyses and PCR amplification of CAG repeats. PCR conditions were same as above, and hSCA7.forward and mSca7.hybrid.reverse (5′-CCACCCACAGATTCCACGAC-3′) were used as primers (FIG. 7B) and PCR amplification of CAG repeats (data not shown).

Both mouse and human ataxin-7 have a nuclear localization signal (NLS) (David et al., 1997; Ström et al., 2002). In order to determine the subcellular localization of ataxin-7 in mouse cerebellum, we isolated the nuclear and cytoplasmic fractions and analyzed them by Western blotting (FIG. 7C). Wild-type ataxin-7 was detected as 93 kDa protein in the nuclear extracts, but not in the cytoplasmic extracts of wild-type or Sca7^{266Q/5Q} mice, showing that it is predominantly a nuclear protein in the cerebellum (FIG. 7C). Mutant ataxin-7 is predicted to migrate at ≧190 kDa, give the size of expanded polyglutamine tract, but no immunoreactivity in this size range was detected in Sca7^{266Q/5Q} lanes. Ataxin-7 immunoreactivity was detected in the stacking gel of the Sca7^{266Q/5Q} nuclear extract lane (FIG. 7C), suggesting that the mutant protein may exist in an insoluble form or in complexes with other proteins.

### Example 10

General Features of Sca7^{266Q/5Q} Mice

Two independent lines of Sca7^{266Q/5Q} mice (lines C1 and H10) were generated. Both lines were indistinguishable from wild-type littermates by visual observation until 5 weeks of age, at which time they started to develop features of infantile SCA7 patients, such as progressive weight loss, droopy eyelids (ptosis), ataxia, muscle wasting curvature of the spine (kyphosis), and tremors (FIG. 7D). CAG repeat size in these two lines of Sca7^{266Q/5Q} mice was relatively stable, occasionally showing less than ten CAG repeat (3o nucleotides) contraction, which did not change the progression or severity of the disease. Sca7^{266Q/5Q} mice showed a similar weight gain pattern as wild-type mice up to 5 weeks but gained little if any weight afterward (FIG. 7E). At the terminal stage of the disease, animals became extremely hypokinetic and did not drink nor eat, even when their chow was wetted and placed at the bottom of the cage. Sca7^{266Q/5Q} mice died around 14-19 weeks of age.

Male Sca7^{266Q/5Q} mice bad reduced fertility after 13 weeks of age. Female Sca7^{266Q/5Q} mice, on the other hand, failed to deliver pups when they were mated after 8 weeks of age. Some Sca7^{266Q/5Q} mice developed myoclonic seizures around 12 weeks of age. Homozygous mice (Sca7^{266Q/5Q} mice) showed a gene dosage effect, with their phenotype being more severe and disease progression more rapid, resulting in death around 7-8 weeks of age. The enhanced toxicity in the homozygous mice is most likely due to higher levels of expanded ataxin-7 (gain-of-function) rather than partial deficiency of wild-type atxin-7's function: mice compound heterozygotes for expanded and a loss-of-function Sca7 alleles (Sca7^{266Q/5Q} mice) do not show enhanced toxicity as those homozygotes for the expanded allele (S. -Y. Y. and H. Y. Z, unpublished data). Because Sca7^{266Q/5Q} mice were so severely affected and because both knockin lines showed similar phenotypes, only Sca7^{266Q/5Q} mice (line H10) were used for subsequent characterization.

### Example 11

Cone-Rod Dystrophy in Generation of Sca7^{266Q/5Q} Mice

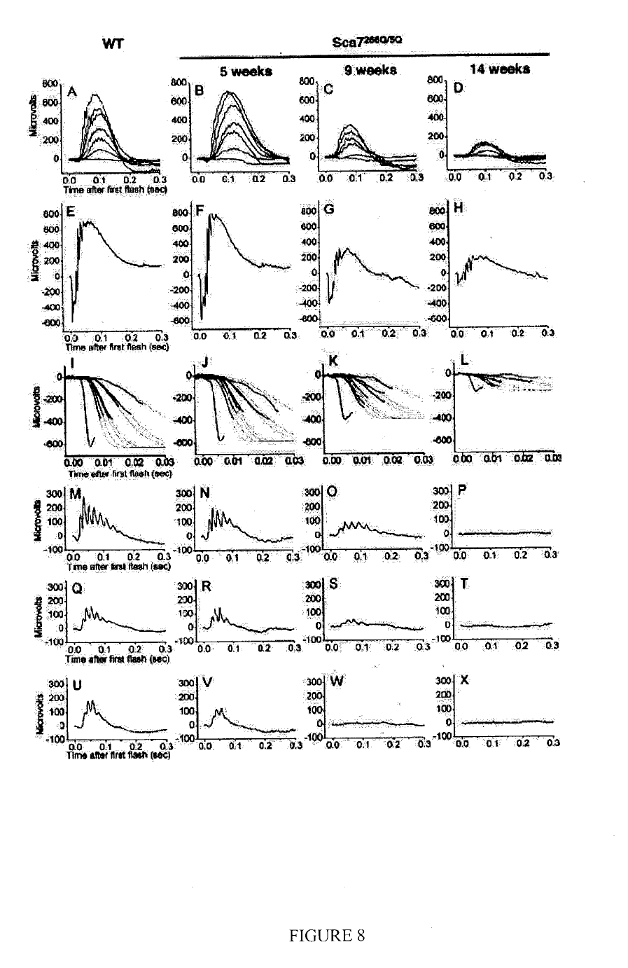

Sca7^{266Q/5Q} mice were born with normal eyes, but as they aged, the eyes receded and ptosis developed. In order to determine whether this was accompanied by a progressive decline of retinal function, we evaluated electroretinograms (ERGs) of wild-type and Sca7^{266Q/5Q} mice at three different ages: 5, 9, and 14 weeks. The ERGs of wild-type mice from these age groups were not significantly different, so we pooled the data for all wild-type animals. We measured scotopic b-waves, a-waves, and cone-driven b-waves (Table 5; FIG. 8).

TABLE 5
Summary of ERG Parameters

| | | | I_{0.5} | | kA | | | |
|---|---|---|---|---|---|---|---|---|
| | Age | b_{max,scot} | (scotopic cd- | aₘₐₓ | s⁻²(scotopic | b_{max,phot} | b_{365 nm} | b_{500 nm} |
| Genotype | (weeks) | (μV) | s/m²) | (μV) | cd-s/m²)⁻¹ | (μV) | (μV) | (μV) |
| Wild- | 5-14 | 625 ± 10 | (14.6 ± 1.3) × | 610 ± 145 | 3360 ± 1610 | 185 ± 35 | 125 ± 35 | 150 ± 40 |
| type | | (n = 10) | 10⁻⁴ | | (n = 8) | (n = 14) | | |
| Sca7^{266Q/5Q} | 5 | 690 ± 9 | (15.7 ± 1.0) × | 650 ± 120 | 3750 ± 1200 | 180 ± 40 | 115 ± 20 | 120 ± 35 |
| | | (n = 4) | 10⁻⁴ | | (n = 4) | (n = 7) | | |
| Sca7^{266Q/5Q} | 9 | 360 ± 10 | (31.7 ± 3.2) × | 320 ± 50 | 3700 ± 1690 | 75 ± 25 | 35 ± 15 | 15 ± 10 |
| | | (n = 5) | 10⁻⁴ | | (n = 8) | (n = 5) | | |
| Sca7^{266Q/5Q} | 14 | 150 ± 4 | (55.1 ± 5.8) × | 165 ± 110 | 2800 ± 1670 | 0 (n 32 4) | 0 | 0 |
| | | (n = 8) | 10⁻⁴ | | (n = 5) | | | |
| The first column indicates the genotype of the mice. Mice were divided into three age groups: 5, 9, and 14 weeks. The parameter, b_{max,scot}, represents the saturating amplitude of the scotopic b-wave, and I_{0.5} is the intensity, measure scotopic cd-s/m², which produces one-half of b_{max,scot}. Theses values were obtained by measuring the peak amplitude of filtered b-waves at increasing intensity and then fitting these values to a hyperbolic saturating function | | | | | | | | |
| # (Naka-Rushton). The value shown in the table represents the best fit, while the second numbers is the error of the fit. for the remaining parameters in the table, the first value represents the mean and the second value represents the standard error. The parameter, aₘₐₓ, is the amplitude of the a-wave in response to an intense, saturating flash measured to produce 2.97 log scotopic cd-s/m². In a photoreceptor with a normal outer segment, this flash is estimated to produce | | | | | | | | |
| # 880,000 photoisomerizations/rod. The parameter kA was obtained by fitting the a-wave using the model developed by Lamb and Pugh. b_{max,phot} measures the peak amplitude of the filtered b-waves in response to a flash that produces 143 photopic cd/m². The parameters b₃₆₅ₙₘ and b₅₀₀ₙₘ are the cone-isolated responses to these flashes, which produce 6,500 and 8,800 photoisomerizations/cone, respectively. | | | | | | | | |

The scotopic (dark-adapted) b-waves is an extracellular field potential that arises primarily from ON-rod bipolar cells in response to dim flashes of light (Pugh et al., 1998). The relationship between scotopic b-wave amplitude and intensity can be modeled using a hyperbolic saturation function. Flashes for scotopic measurements were generated by a Grass PS-33+ photostimulator. Light was spectrally filtered with a 500 nm interference filter (Edmund Scientific). A series of metal plates with holes of varying diameters and glass neutral density filters were used to attenuate the flash. As the intensity of the flash increased, the number of trials was decreased and the time between each flash was increased. To remove oscillatory potentials prior to fitting, the scotopic b-wave was digitally filtered using the filtfilt function in Matlab (low pass filter, Fc=60 Hz). To calculate b_{max,scot} and I0.5, the peak value of each curve was fit to a saturating hyperbolic function (Naka-Rushton). For analysis of the a-wave and cone function, 1500 W Novatron xenon flash lamps provided intense illumination. To analyze the rod function, we used the following equation (Lamb-Pugh model) to fit a series of a-waves at increasing intensities:

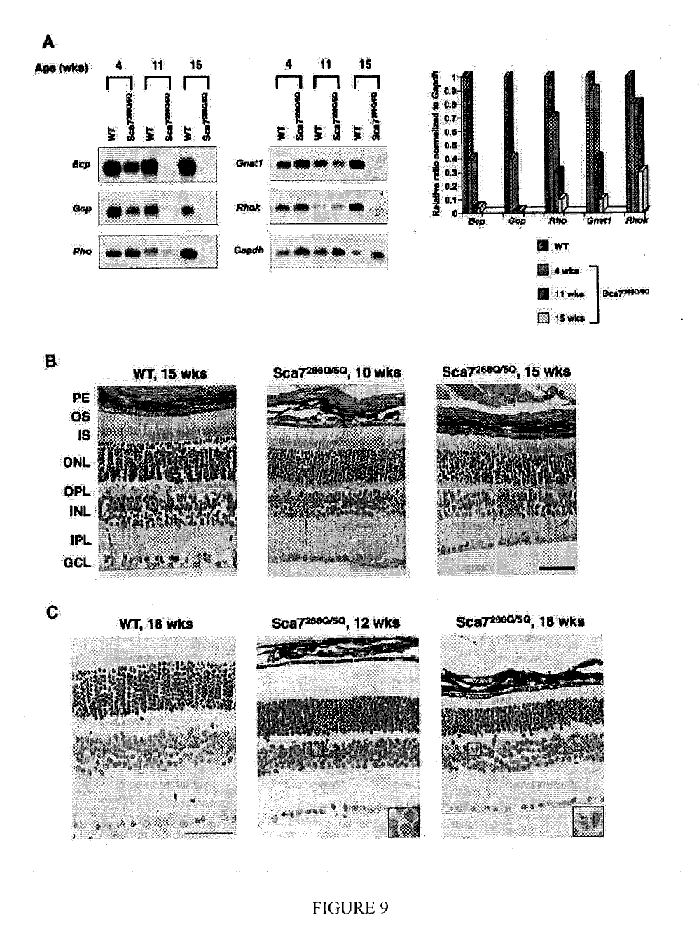

1−a(t)/aₘₐₓ=exp [−½·ø·A·(t−t_{eff})²] Equation 1

where a(t) is the a-waive, aₘₐₓ is its saturating amplitude, ø is the number of photoisomerizations rod produced by the flash, A is amplification factor, and t_{eff} is a brief delay. Because pathological studies indicated that the outer segments were shorter in Sca7^{266Q/5Q} mice (FIG. 9B), we substituted ø=I k into the equation, and fit for the parameter kA (Hood and Birch, 1994). I is intensity in scotopic cd/m2 and k is variable with unit ø/scotopic cd/m2. Cone-driven responses were recorded using the paired flash technique. The first flash drove the rods temporarily into saturation. The second flash, delivered 2.5 s later, allowed the cones to recover, but not the rods. To isolate responses from S cones or M cones, we used bandpass interference filters centered at 365 and 500 nm (Edmund Scientific), respectively. To remove oscillatory potentials prior to fitting, the cone b-waive was digitally filtered using the filtfilt function in Matlab (low-pass filter, Fe=30 Hz).

This model yields two parameters, b_{max,sscot} and 10.5, representing the maximum b-wave amplitude and the intensity that provides half saturation, respectively. At 5 weeks of age, scotopic b-waves from Sca7^{266Q/5Q} mice demonstrated both a decline in b_{max,sscot} and an increase in 10.5 (Table 5). At 9 weeks, rod-driven function had decreased to 65% compared to wild-type (FIGS. 8A-8D). It was not possible to study the mutant mice beyond 15 weeks because of their frailty. Our data suggest that the rod system of Sca7^{266Q/5Q} mice develops properly and is capable of normal phototransduction. The decrease in the scotopic b-wave may have resulted from a defect in the rod bipolar cells, from decreased input of the rod photoreceptors, or both.

To examine rod photoreceptor function, the ERG a-wave was analyzed, which arises almost exclusively from the rod photoreceptors in mouse (Pugh et al., 1998). An intense flash was used to measure the saturated a-wave amplitude, aₘₐₓ (FIGS. 8E-8H). At 5 weeks of age, there was no significant different between the wild-type and Sca7^{266Q/5Q} mice. Sca7^{266Q/5Q} mice showed a steady decline in saturated a-wave amplitude with age: at 9 weeks, aₘₐₓ had declined to 60%, and by 14 weeks, aₘₐₓ was decreased to 30% of normal. To analyze the amplification of the rod transduction cascade, a series of a-waves were fitted to the above equation (Lamb and Pugh, 1992), and the parameter kA was derived (FIGS. 81-8L). There was no statistically significant different in the amplification parameter kA between wild-type and Sca7^{266Q/5Q} mice at any of the ages tested (Table 5). The amplitude of the a-wave is directly proportional to the number of cyclic nucleotide-gated channels that close in response to light (Pugh et al., 1998). Thus, the decrease of the a-wave in Sca7^{266Q/5Q} mice provides direct evidence for a rod photoreceptor defect. The normal amplification constant suggests that this deficit is not due to a change in the relative enzymatic activities of the proteins involved in the transduction cascade. A loss of photoreceptors or decrease in the photoreceptor outer segment length could diminish the a-wave amplitude without necessarily changing the amplification constant. To determine whether the dysfunction extended to cones, the double flash technique was used to isolate responses from cone photoreceptors (Lyubarsky et al., 1999). FIGS. 8M-8P show the ERG response to a white flash, which stimulated both S and M cones. To establish whether there was any functional different between S and M cones, the light was filtered using band pass filters specific for the two classes of cones and measured the response (FIGS. 8Q-8T and 8U-8X). At 5 weeks wild-type and Sca7^{266Q/5Q} mice showed little different when stimulated with white or UV light, but stimulation with green light revealed a statistically significant decrease (p=0.039) (Table 5; FIG. 8V). The cone response continued to decreased at 9 weeks, and by 14 weeks, it was undetectable (FIGS. 8P, 8T, and 8X). The trend toward a decline at 5 weeks and the significant decline at 9 weeks of come ERGs suggests that cone dysfunction occurs prior to rod dysfunction in Sca7^{266Q/5Q} mice.

The abnormal ERG results prompted us to look at the expression of levels of several photoreceptor-specific genes in the eyes of Sca7^{266Q/5Q} mice (FIG. 9A). The expression levels were examined of three photopigment genes (blue cone opsin [Bcp], green cone opsin [Gcp], and rhodopsin [Rho]), rod transducin (Gnat1), and rhodopsin kinase (Rhok), Bcp (S opsin) and Gcp (M opsin) are cone-specific markers, whereas Rho and Gnat1 are rod-specific markers. Rhok is present in both cones and rods (Lyubarsky et al., 2000). Total RNA was isolated from the mouse eyes and brain with TRIZOL reagent following manufacture's instructions (Invitrogen). For expression analyses of photoreceptor-specific genes, 10 μg of total RNA from eyes was loaded on each lane for electrophoresis and transferred onto Hybond N+ nylon membrane (Amersham). All probes were obtained from the mouse EST clones (ATCC): Bcp (GenBank accession number BG293946, 295-818 bp Pstl/Nhel fragment), Gcp (GenBank accession number BG297657, 11-629 bp Ncol/Kpnl fragment), Rho (GenBank accession number BG296640, 73-423 bp Xhol fragment), Gnat1 (GenBank accession number BG404720, 68-646 bp BstXI/BamHI fragment), Rhok (GenBank accession number BG296640, 842-1387 bp Kpnl/EcoRV fragment), Rom1 (GenBank accession number BG404693, 9-515 bp Nar1 fragment), Rbp3 (GenBank accession number BI737899, 846-1200 bp Apal/BamHI fragment), and Cnga3 (GenBank accession number BI730059, 537-1364 bp, Smal fragment). For Sca7 expression analyses, 15 μg of total RNA from brain was loaded on each lane for electrophoresis and transferred onto Hybond N+ nylon membrane (Amersham). Probe was isolated from mouse Sca7 cDNA (360-912 bp HindIII/Pstl fragment). Probes were labeled using Megaprime kit (Amersham) and hybridized in ExpressHyb hybridization solution (Clontech) according to manufacture's instructions. The intensity of each band was measured by densitometry. Expression of all these genes showed a progressive decreased with age. Cone-specific markers showed about 60% reduction in a presymptomatic 4-week-old mutant (FIG. 9A). The youngest mice that were tested on ERGs were 5 weeks old when they began to show some decrease in cone activity (Table 5; FIG. 8). It is interesting to note that the cone photoreceptors remained functional even with more than 60% decrease of both cone-specific photopigments. Rod-specific markers were downregulated later than cone-specific markers. This may explain why cone dysfunction appeared earlier than rod dysfunction in ERG studies.

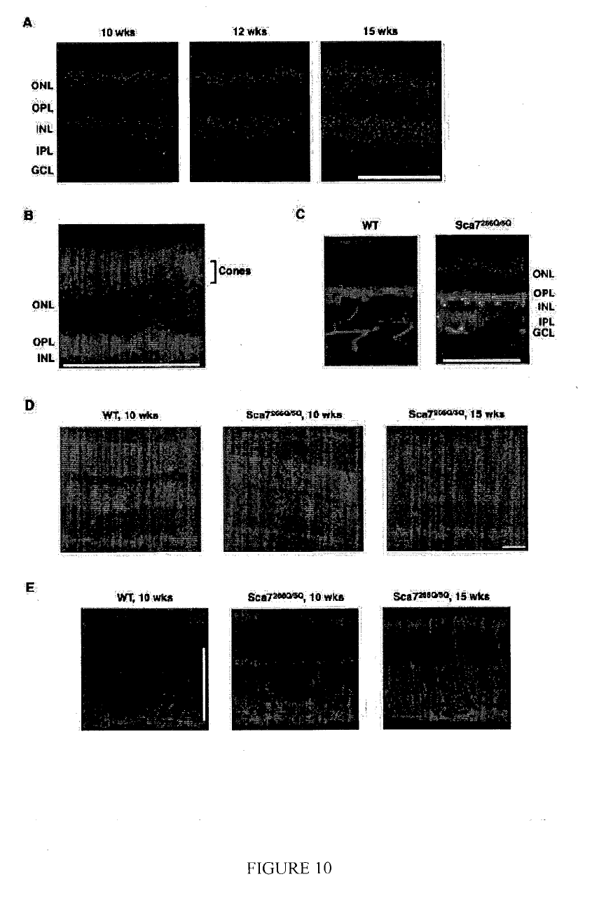

Hematoxylin and eosin staining of Sca7^{266Q/5Q} retinas revealed progressive shortening of outer segments and thinning of inner plexiform layer (IPL) (FIG. 9B). Although endogenous levels of ataxin-7 were very low in wild-type retina, mutant ataxin-7 accumulated in Sca7^{266Q/5Q} retina, forming microaggregates around 8 weeks of age (Table 6)—and single, large nuclear inclusions (Nis) around 10 weeks (FIGS. 9C and 10A)—when retinal dysfunction has already set in. Mutant ataxin-7 accumulation was observed throughout the retina, but it occurred more rapidly in outer nuclear layer (ONL) (specifically the top layer of ONL) and inner nuclear layer (INL) than in ganglion cell layer (GCL) (FIGS. 9C and 10A). In mature mouse retina, the nuclei of cones are aligned directly beneath the inner segments, i.e., in the top layer of ONL (Carter-Dawson and LaVail, 1979; Rich et al., 1997). To verify that the nuclei harboring mutant ataxin-7 signals in the ONL of 10-week-old Sca7^{266Q/5Q} retina are those of cones, retina sections were double-labeled with ataxin-7 antibody (1261) in combination with neuron-specific enolase (NSE) antibody, which labels cones (Rich et al., 1997). All ataxin-7 positive nuclei in ONL, at this age, turned out to be those of cones, suggesting that accumulation of mutant ataxin-7 starts in the cones in the ONL (FIG. 10B). Double labeling using 1261 antibody was also performed in combination with either calbindin antibody (a marker for horizontal cells and amacrine cells) or protein kinase C (PKC) antibody (a marker for bipolar cells) on 10-, 12-, and 15-week-old retina sections (FIG. 10C and data not shown) and confirmed that the mutant protein accumulated in nuclei of all three interneurons in INL.

TABLE 6
Time-Dependent Appearance of NIs in the Brain and Retina

| | NI^{a} | Ub- | Hsc70- | CBP- | TBP- |
|---|---|---|---|---|---|
| | Appear- | Positive | Positive | Positive | Positive |
| | ance | Staining | Staining | Staining | Staining |
| Area | (Weeks) | (Weeks) | (Weeks) | (Weeks) | (Weeks) |
| Spinal Cord | ≧5 | ≧12 | ≧12 | ≧18 | — |
| Pons/Medulla | ≧5 | ≧12 | ≧12 | ≧18 | — |
| Olfactory bulb | ≧7 | ≧15 | ≧15 | ≧18 | — |
| Cortex | ≧5 | ≧12 | ≧15 | ≧18 | — |
| Cerebellum | ≧12 | ≧12 | ≧12 | ≧18 | — |
| Hippocampus | ≧13 | ≧13 | ≧13 | ≧18 | — |
| Retina | ≧8 | ≧12 | ≧12 | — | — |

^{a} Here used to denote any visible aggregation, including microaggregates.
^{b} In these tissues, NIs appeared in glial cells at the designated ages.

Although NIs were present in all three nuclear layers in the retina, apoptosis was observed only in ONL (FIG. 10D). More importantly, retinal dysfunction occurred prior to the loss of photoreceptors: both rod and cone activities were significantly reduced at 9 weeks (FIG. 8), but only few photoreceptors were TUNEL positive in ONL of 10-week-old Sca7^{266Q/5Q} mice (FIG. 10D). To quantify the loss of photoreceptors, the number of nuclei was counted in the vertical length of ONL at 15 weeks of age. Briefly, eyes of three mice were used for each genotype (five section from the center/mouse). Sections were cut in 5 μm thickness. Pictures were taken at 20× magnification using a Zeiss Axioplan 2 fluorescence microscope and Axiocam camera following Hematoxylin and eosin staining. The number of vertical nuclei in ONL was counted every 25 μm on these pictures (five to six counts were performed/picture). Mean, SEM, and t test (two sample assuming unequal variance) were determined in Microsoft Excel. Sca7^{266Q/5Q} mice lost about 20% of photoreceptors in the ONL at this age (WT, 10.9±0.1; Sca7^{266Q/5Q}, 8.8±0.11; p<0.0001). Progressive activation of Miller glia in Sca7^{266Q/5Q} retina was also observed, which is a good indicator for photoreceptor degeneration (Semple-Rowland et al., 1991; de Raad et al., 1996; Hong et al., 2000; Rattner et al., 2001) (FIG. 10E). The results indicate that the abnormal ERGs in Sca7^{266Q/5Q} mice are not due to the loss of photoreceptors, but to photoreceptor dysfunction and transcriptional dysregulation of photoreceptor-specific genes.

### Example 12

Cerebellar Dysfunction in Sca7^{266Q/5Q} Mice

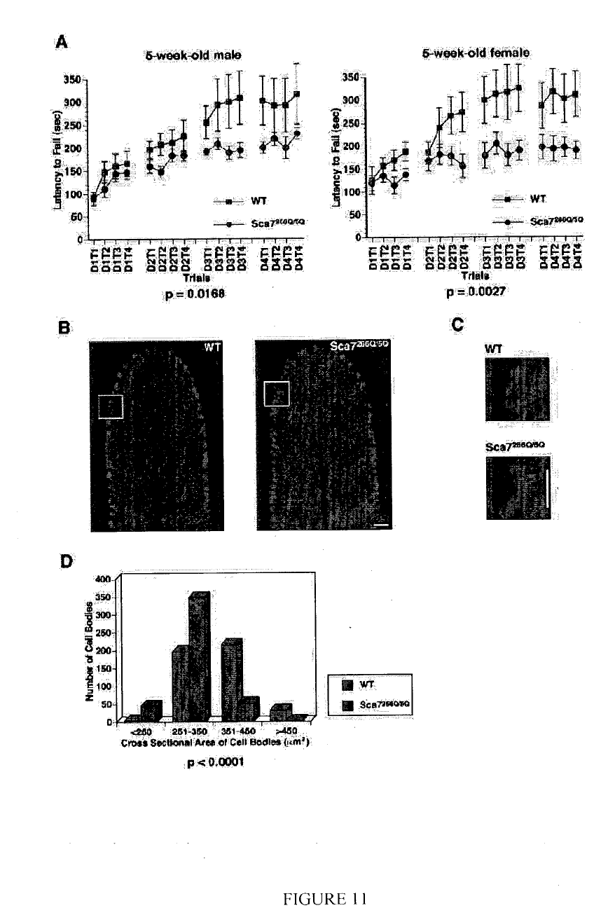

In addition to the retina, the cerebellum is one of the most commonly affected tissues in infantile SCA7. Sca7^{266Q/5Q} mice manifested motor incoordination on the rotarod by 5 weeks. Although their rotarod performance improved with training, they did not last as long on the rotarod as their wild-type littermates (FIG. 11A). By 8-9 weeks, gait ataxia was apparent and motor coordination deteriorated further. Mutant mice soon became kyphotic, then hypoactive at the terminal stage of disease. Pathologic inspection of Sca7^{266Q/5Q} brain revealed that it was smaller than wild-type. TUNEL assays at 15 and 18 weeks detected no positive signals in cerebellum (data not shown). To perform the TUNEL assay, eyes were fixed in 5% PFA/PBS for overnight at room temperature and cryoprotected by infiltrating from 15% sucrose/PBS to 30% sucrose/OCT (Tissue-Tek). They were subsequently embedded in OCT and cut as 10 μm sections onto Superfrost/Plus slides (Fisher Scientific). TUNEL assays were performed using in situ cell death detection kit, AP following manufacture's protocol (Roche). Images were acquired by a Zeiss Axioplan 2 fluorescence microscope and Axiocam camera.

In order to evaluate whether there was any loss of Purkinje cells in Sca7^{266Q/5Q} cerebellum at the terminal stage, the number of Purkinje cells were counted in a 5 μm section through the midsagittal cerebellar region at 18 weeks. Brains of three mice were used for each genotype. Purkinje cells on three to six serial midsagittal cerebella hemisphere 5 μm sections (multiple sections/brain) were counted following Hematoxylin and eosin staining. Mean, SEM, and t test (two sample assuming equal variance) were determined in Microsoft Excel. No significant Purkinje cell loss was detected in the mutants (WT, 273.3±5.8; Sca7^{266Q/5Q}, 264.3±8.6; p=0.2), which is consistent with the findings in some infantile SCA7 cases (Carpenter and Schumacher, 1966). Immunofluorescence confocal microscopy revealed that although the number of Purkinje cells and their dendritic arbors appeared normal in 16-week-old Sca7^{266Q/5Q} cerebellum, the cell bodies were significantly smaller than those of wild-type Purkinje cells (FIGS. 11B and 11C). Briefly, 15 μm stacks of cerebellar optical sections were collected using a Zeiss 510 confocal microscope and averaged using the z projection function of NIH Image J. From three projected stacks (150 cells each) for each of three wild-type and three Sca7^{266Q/5Q} mice, a total of 450 wild-type and 450 Sca7^{266Q/5Q} cells were outlined manually and the cross-sectional area measured. Partial cells and binary cell images were excluded based upon cell shape and relative fluorescence intensity. Perikaryon area was estimated to be the approximately circular area enclosed by the cell perimeter and the extension of the cell perimeter toward the initial point of dendrite extension.

### Example 13

Mutant Ataxin-7 is Highly Insoluble and Gradually Accumulates in Neurons

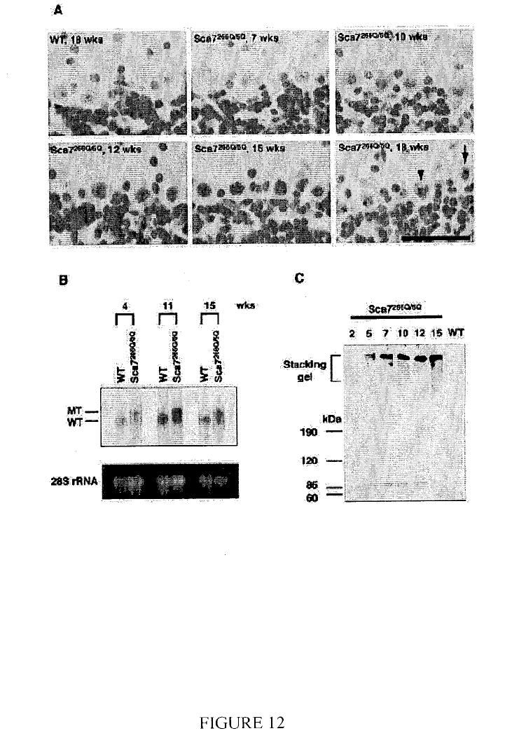

No ataxin-7 immunostaining was apparent in wild-type brain sections with the 1261 antibody made against amino acid 1-19 of human ataxin-7 (kind gift of J. -L. Mandel [Lindenberg et al., 2000; Yvert et al., 2000]) and SCA7 (1-135) antibody made against amino acid 1-135 of mouse ataxin-7 (kind gift of M. Holmberg [Ström et al., 2002]) (FIG. 12A and data not shown). The same antibodies were, however, able to detect mutant ataxin-7 in Sca7^{266Q/5Q} brain, and the staining became denser as the animal aged (Table 6; FIG. 12A). The pattern of immunoreactivity varied, however, in different neurons. NIs did appear in nonaffected areas (areas that are typically spared in infantile SCA7 patients [Havener, 1951; Carpenter and Schumacher, 1966]) first; however, they were in glial cells and not in neurons. Neurons of the retina and cerebellum, two areas whose functions are profoundly affected in infantile SCA7 patients and Sca7^{266Q/5Q} mice, showed faster accumulation of mutant ataxin-7 than other areas of the brain in Sca7^{266Q/5Q} mice. It is interesting that although mutual ataxin-7 accumulation increased over time, NIs were not formed until much later in the disease course (10-12 weeks) in these two affected areas (FIGS. 9C, 10A, and 12A). This accumulation of mutant ataxin-7 was not due to the upregulation of mutant Sca7 mRNA, since there was no difference between the level of mutant and wild-type Sca7 mRNA in the mutant brain (FIG. 12B).

The hippocampus was another area where accumulation of mutant ataxin-7 in neurons was occurring around the same time as the retina and cerebellum, which prompted a search for functional impairments specific to this tissue (see below). It is noteworthy that in three areas that develop functional impairments in Sca7^{266Q/5Q} mice (retina, cerebellum, and hippocampus), the aggregates became ubiquitin positive around 12 weeks (Table 6). Ubiquitin-positive NIs colocalized with Hsc70, and the number of ubiquitin- and Hsc70-positive NIs increased over time (data not shown). No CRX or TATA binding protein (TBP) immunoreactivity was observed in NIs of affected neurons up to 18 weeks (Table 5). NIs throughout the brain became CREB binding protein (CBP) positive around 18 weeks (Table 5).

Wild-type ataxin-7 was detected only in nuclear extracts, but not cytoplasmic extracts of wild-type and Sca7^{266Q/5Q} cerebella (FIG. 7D). There was no detection of any SDS-soluble mutant ataxin-7 or any evidence of truncated mutant ataxin-7 using N-terminal (1261) and C-terminal (1597) (Yvert et al., 2000) antibodies in the separating gel (FIG. 7C and data not shown). Mutant ataxin-7 was detected only in the stacking gel, suggesting that this protein exists as an insoluble multimer or in complexes with other proteins. Another possibility is that the conformation of soluble mutant ataxin-7 differs from that of insoluble mutant, ataxin-7, and the epitope of soluble mutant ataxin-7 is masked. It was reasoned that because the mutant protein accumulated with age, it may be possible to detect the soluble form in younger animals. Cerebellar nuclear extracts were prepared from 2-, 5-, 7-, 10-, 12-, and 15-week-old Sca7^{266Q/5Q} mice, and Western blotting was performed using the 1261 antibody. Western blotting was performed as follows. Nuclear and cytoplasmic extracts of mouse cerebellum were obtained using NE-PER Nuclear And Cytoplasmic Extraction Reagents (Pierce). 100 μg of protein was loaded on 6% SDS gel. Dilution factor for 1261 was 1:1000. For expression analyses of CRX, retinal protein extracts were prepared by homogenizing mouse retinas in buffer containing 100 mM Tris (pH 6.8), 25mM DTT, 2% SDS, and 2× complete protease inhibitor cocktail (Roche). 20 μg of extracts were loaded on 10% SDS gel. Dilution factor for CRX antibody was 1:500.

Expression levels of wild-type ataxin-7 were very low in both wild-type and Sca7^{266Q/5Q} mice, and mutant ataxin-7 immunoreactivity in the stacking part of the gel increased with age. Surprisingly, the soluble mutant ataxin-7 was not detected even in 2-week-old mice when very faint immunoreactivity was found in the stacking gel. This suggests that there is little accumulation of mutant ataxin-7 at this age. These data indicate that even in 2-week-old Sca7^{266Q/5Q} cerebellum, soluble mutant ataxin-7 might exist in a conformation that is not easily detectable with 1261 antibody or that it is still efficiently turned over. By 5 weeks, however, mutant ataxin-7 begins to accumulate, becoming part of insoluble complexes.

### Example 14

Selective Impairment of PTP

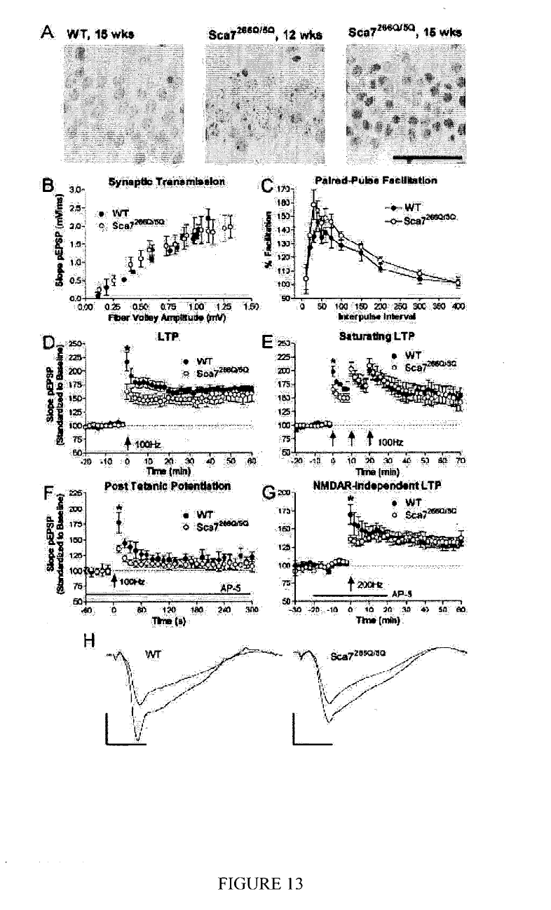

Hippocampal function was investigated in Sca7^{266Q/5Q} mice, since the hippocampus is another area in which mutant ataxin-7 rapidly accumulated in neurons (Table 5; FIG. 13A). Occasional punctate nuclear immunostaining of ataxin-7 was observed in hippocampal CA1 pyramidal neurons around 12weeks, and by 15 weeks, NIs became larger and dotted the entire hippocampus (FIG. 13A and data not shown). It was investigated whether synaptic physiology was altered at the time of mutant ataxin-7 accumulation in hippocampal area CA1. Hippocampal slices were prepared from 12-week-old Sca7^{266Q/5Q} mice and littermate controls and characterized synaptic transmission and short- and long-term plasticity using extracellular field recordings in stratum radiatum in area CA1. Hippocampal slice preparation and electrophysiology were performed as previously described (Roberson and Sweatt, 1996). Briefly, hippocampal slices (400 μm) were maintained in an interface chamber and bathed in oxygenated artificial cerebral spinal fluid (ACSF, 125 mM NaCl, 2.5 mM KCI, 1.25 mM NaH₂PO₄, 25 mM NaHCO₃, 10 mM D-glucose, 2 mM CaCl₂ and 1 mM MgCl₂) (1 ml/min) at 30° C. Extracellular field recordings were made every 20 s in area CA1 stratum radiatum during stimulation of the Schaffer collateral pathway. Stable baseline synaptic transmission was established for 20 min at an intensity of 40%-50% of the maximum population excitatory postsynaptic potential (pEPSP) prior to LTP-inducing high-frequency stimulation (HFS). Stimulus intensity of the HFS was matched to the intensity used in the baseline recordings, and measurements are shown as the average slope of the pEPSP from six individual traces standardized to baseline recordings. LTP was induced by one or three sets of HFS, each set consisting of two trains of 100 Hz stimulation for 1 s, separated by 20 s (NMDA-dependent LTP induction), or a single train of 200 Hz stimulation for 1 s (NMDA-independent LTP induction). PTP was induced by a single 100 Hz HFS train for 1 s following at least 20 min of stable baseline recordings in the presence of AP-5. Immediately after the HFS, pEPSPs were recorded every 3 s for 5 min and are presented as the average of four individual traces. No difference was detected in baseline synaptic transmission, as input-output functions for increasing stimulus intensities in Sca7^{266Q/5Q} mice are similar to wild-type (FIG. 13B). Normal baseline synaptic transmission suggests that the synaptic connectivity in Sca7^{266Q/5Q} mice is normal. Paired pulse facilitation (PPF), a form of short-term synaptic plasticity, was slightly elevated in Sca7^{266Q/5Q} mice, with the greatest difference at 30 ms interpulse interval (Sca7^{266Q/5Q}, 158% ±1%; WT, 135% ±4%), but PPF across all interpulse intervals did not show statistically significant differences (FIG. 13C).

Synaptic enhancement of the Shaffer collateral synapse following high-frequency stimulation (HFS) can be divided into three distinct phases: (1) initial LTP (I-LTP) (commonly referred to as short-term potentiation [STP]), (2) early LTP (E-LTP), and (3) late LTP (L-LTP) (Sweatt, 1999). Sca7^{266Q/5Q} mice showed a significant reduction in I-LTP (indicated by the asterisk in FIG. 13D), following the LTP-including HFS using a standard two trains of 100 Hz stimulation (FIG. 13D), but normal E-LTP. We tested whether the same LTP impairment could be seen in Sca7^{266Q/5Q} mice under the saturating HFS paradigm. LTP was induced in Sca7^{266Q/5Q} mice with three pairings of 100 Hz stimulation (6×, 100 Hz trains total) (FIG. 13E). This clearly shows that Sca7^{266Q/5Q} mice are capable of maximum potentiation under saturating HFS. Consistent with our previous results, there was a significant reduction in I-LTP following the first pairing of stimulation, but the deficit in I-LTP could be rescued with successive pairings of 100 Hz stimulation (FIG. 13E). A prominent component of I-LTP is posttetanic potentiation (PTP), a short-lived form of presynaptic plasticity immediately following HFS, characterized by greatly enhanced potentiation lasting for 1-5 min. In order to isolate the PTP component from I-LTP, the NMDA receptor antagonist 2-amino-5-phosphonovaleric acid (AP-5) was used to mask postsynaptic potentiation but leave PTP intact. Using this protocol, it was found that the changes in I-LTP in Sca7^{266Q/5Q} mice were due in part to a deficit in PTP (FIG. 13F). NMDA receptor-independent LTP was also tested in Sca7^{266Q/5Q} mice. Similar to the NMDA receptor-dependent LTP results, there was no difference in NMDA receptor-independent LTP between wild-type and Sca7266Q/5Q mice, and again a significant reduction in the first few minutes of LTP was observed (FIG. 13G). Our data suggest that the I-LTP impairment in Sca7^{266Q/5Q} mice is not restricted to stimulation in the 100 Hz frequency range, but is observed at higher stimulation frequencies (200 Hz) as well. Deficits in Sca7^{266Q/5Q} I-LTP or PTP do not appear to be due to changes in baseline synaptic responses since EPSP wave forms from CA1 field recordings were indistinguishable from those of controls (FIG. 13H).

1. A knock-in mammal containing integrated into its genome a repeating polyglutamine sequence comprising at least 154 contiguous codons encoding glutamine and exhibiting at least one phenotype characteristic associated with a neurodegenerative disease.

2. The knock-in mammal of claim 1 wherein said mammal demonstrates one or more of the phenotype characteristics shown in Table 3.

3. The knock-in mammal of claim 1 wherein said mammal demonstrates accumulation of a protein comprising at least about 154 contiguous glutamine residues in neurons.

4. The knock-in mammal of claim 1 wherein said mammal demonstrates clinical symptoms of a disease selected from the group consisting of spinobulbar muscular atrophy (SBMA), Huntington's disease (HD), dentatorubral pallidoluysian atrophy (DRPLA), and the spinocerebellar ataxias types 1, 2, 3, 6, 7, and 17.

5. The knock-in mammal of claim 1, wherein said mammal is a rodent.

6. The knock-in mammal of claim 5, wherein said rodent is a mouse.

7. The knock-in mammal of claim 1, wherein the at least 154 codons comprise the codon CAG.

8. The knock-in mammal of claim 1, wherein the contiguous codons encoding glutamine are present in a spinocerebellar ataxia gene.

9. The knock-in mammal of claim 8 wherein said spinocerebellar ataxia gene is selected from the group consisting of Sca1, Sca2, Sca3, Sca7, and Sca17.

10. The knock-in mammal of claim 1, wherein the contiguous codons encoding glutamine are present in a genetic locus selected from the group consisting of Xq13-21, 4p16.3, 12p13.31, 6p23, 12q24.1, 14q32.1, 3p12-13, and 6q27.

11. The knock-in mammal of claim 1, wherein the contiguous codons encoding glutamine are present in the coding sequence of a protein selected from the group consisting of an androgen receptor, huntingtin, atrophin-1, ataxin-1, ataxin-2, ataxin-3, ataxin-7, and a TATA-binding protein.

12. The knock-in mammal of claim 1, wherein the contiguous codons encoding glutamine are present in exon 8 of a Sca1 gene.

13. The knock-in mammal of claim 1, wherein the contiguous codons encoding glutamine are present in exon 3 of a Sca7 gene.

14. The knock-in mammal of claim 1 wherein the contiguous codons encoding glutamine are introduced into the genome of the mammal by a construct comprising a portion of a Sca1 exon 8.

15. The knock-in mammal of claim 1, wherein the contiguous codons encoding glutamine are introduced into the genome of the mammal by a construct comprising a portion of a Sca7 exon 3.

16. A knock-in mammal containing integrated into its genome a repeating polyglutamine sequence comprising at least 154 contiguous codons encoding glutamine and exhibiting at least one phenotype characteristic associated with a neurodegenerative disease, and wherein said repeating polyglutamine sequence is integrated into a gene selected from the group consisting of a spinocerebelar ataxia gene, an androgen receptor gene, a Huntington's disease gene, and a DRPLA gene.

17. The knock-in mammal of claim 16, wherein said spinocerebellar ataxia gene is selected from the group consisting of Sca1, Sca2, Sca3, Sca7, and Sca17.

18. The knock-in mammal of claim 16, wherein said spinocerebellar ataxia gene encodes a protein selected from the group consisting of ataxin-1, ataxin-2, ataxin-3, ataxin-7, and TATA-binding protein.

19. The knock-in mammal of claim 16, wherein said contiguous codons are integrated into a chromosomal locus selected from the group consisting of Xq13-21, 4p16.3, 12p13.31, 6p23, 12q24.1, 14q32.1, 3p12-13, and 6q27.

20. The knock-in mammal of claim 16 wherein said mammal demonstrates accumulation of a protein comprising at least about 154 glutamine residues in sequence in neurons.

21. The knock-in mammal of claim 16 wherein said mammal demonstrates clinical symptoms of a disease selected from the group consisting of the spinocerebellar ataxias types 1, 2, 3, 6, 7, and 17.

22. The knock-in mammal of claim 16, wherein said mammal demonstrates clinical symptoms of a disease selected from the group consisting of Huntington's disease, spinobulbar muscular atrophy, and dentatorubralpallidoluysian atrophy.

23. The knock-in mammal of claim 16 wherein said mammal is a rodent.

24. The knock-in mammal of claim 23, wherein said rodent is a mouse.

25. The knock-in mammal of claim 16 wherein the at least 154 contiguous codons comprise the codon CAG.

26. A knock-in mammal containing integrated into a chromosomal locus selected from the group consisting of Xq13-21, 4p16.3, 12p13.31, 6p23, 12q24.1, 14q32.1, 3p12-13, and 6q27, a repeating polyglutamine sequence comprising at least 154 contiguous codons encoding glutamine and exhibiting at least one phenotype characteristic associated with a neurodegenerative disease.

27. A knock-in mammal containing integrated into a gene which encodes a protein selected from the group consisting of androgen receptor, huntingtin, atrophin-1, ataxin-1, ataxin-2, ataxin-3, ataxin-7, and TATA-binding protein, a repeating polyglutamine sequence comprising at least 154 contiguous codons encoding glutamine and exhibiting at least one phenotype characteristic associated with a neurodegenerative disease.

28. The knock-in mammal of claim 26 or 27, wherein said mammal demonstrates accumulation of a protein comprising at least about 154 glutamines in sequence in neurons.

29. The knock-in mammal of claim 26 or 27, wherein said mammal demonstrates clinical symptoms of a disease selected from the group consisting of spinobulbar muscular atrophy (SBMA), Huntington's disease (HD), dentatorubral pallidoluysian atrophy (DRPLA), and the spinocerebellar ataxias types 1, 2, 3, 6, 7, and 17.

30. The knock-in mammal of claim 26 or 27, wherein said mammal is a rodent.

31. The knock-in mammal of claim 30, wherein said rodent is a mouse.

32. The knock-in mammal of claim 26 or 27, wherein the at least 154 contiguous codons comprise the codon CAG.

33. A method for identifying an agent for treating a disease characterized by the abnormal presence of a polyglutamine sequence in a peptide or protein comprising the steps of:
(a) administering a candidate agent to a knock-in mammal having integrated into its genome a repeating polyglutamine sequence comprising at least 154 contiguous codons encoding glutamine and exhibiting at least one phenotype characteristic associated with a neurodegenerative disease; and
(b) monitoring said mammal to determine the effect of said agent on one or more phenotypic characteristic, wherein if said agent alters said at least one phenotype characteristic, then said agent is identified as an agent for treating a disease characterized by the abnormal presence of a polyglutamine sequence in a peptide or protein.

34. The method of claim 33 wherein the neurodegenerative disease is selected from the group consisting of spinobulbar muscular atrophy (SBMA), Huntington's disease (HD), dentatorubral pallidoluysian atrophy (DRPLA), and the spinocerebellar ataxias types 1, 2, 3, 6, 7, and 17.

35. The method of claim 33 wherein the neurodegenerative disease is a spinocerebellar ataxia.

36. The method of claim 33 wherein said mammal demonstrates one or more of the phenotype characteristics shown in Table 3.

37. The method of claim 33 wherein said mammal demonstrates accumulation of a protein comprising at least 154 glutamines in sequence in neurons.

38. The method of claim 33 wherein said mammal is a rodent.

39. The method of claim 38, wherein said rodent is a mouse
